(19) United States
(12) Patent Application Publication
(10) Pub. No.: US 2004/0197786 A1
(43) Pub. Date: Oct. 7, 2004

(54) Method of examining steroid resnponsiveness

(76) Inventors: Yuji Sugita, Tsukuba-shi (JP); Masayuki Heishi, Konohana-ku (JP); Shinji Kagaya, Setagaya-ku (JP); Shigemichi Gunji, Chuo-ku (JP); Gozoh Tsujimoto, Sakyo-ku (JP)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 10/474,079

(22) PCT Filed: Mar. 1, 2002
(86) PCT No.: PCT/JP02/01917

(30) Foreign Application Priority Data
Apr. 5, 2001 (JP) 2001-107557

(51) Int. Cl. C12Q 1/68; G01N 33/53

(52) U.S. Cl. 435/6; 435/7.1

(57) ABSTRACT

RING6 and HLA-DMB are described herein as genes whose expression levels in mononuclear cells greatly differ between a steroid responder group and a poor steroid responder group in atopic dermatitis patients. Specifically, the expression levels of the RING6 and HLA-DMB genes were demonstrated to be reduced in steroid-responsive patients. Using the expression level of such genes in biological samples as markers of steroid responsiveness, the present invention provides a method of testing for steroid responsiveness and a method of screening for compounds that may be used to improve steroid responsiveness.

### TECHNICAL FIELD

The present invention relates to a method of testing for steroid responsiveness.

### BACKGROUND ART

Allergic diseases such as atopic dermatitis are considered to be multifactorial diseases. These diseases are caused by the interaction of many different genes, the expression of which is influenced by many different environmental factors. Thus, determining the specific genes responsible for a specific allergic disease is extremely difficult.

Overexpression or reduced expression of certain genes, or expression of mutated or defective genes, is thought to play a part in allergic diseases. In order to determine the role of gene expression in allergic diseases, it is necessary to understand how genes are involved in triggering disease onset, and how gene expression is altered by external stimulants such as drugs.

Steroids are fast becoming universally recognized as a means of treating allergic diseases. For example, external steroid preparations are effective in treating atopic dermatitis, and inhalation and oral administration of steroids is considered important in the treatment of bronchial asthma. Steroid preparations suppress both the production of inflammatory cytokines and the activity of activated eosinophils through their stimulation of the glucocorticoid receptor (GR). Thus steroids relieve inflammatory symptoms and are thought to aid treatment of allergic diseases.

Although steroids are important tools in the treatment of allergic diseases, some inflammatory symptoms demonstrate little response to their administration. Such a case is referred to as ‘steroid-resistant’. Patients are classified according to a clinical score of their response to the steroid treatment after two weeks (a modified Leicester score). Patients are classified into ‘responders’ (where their score improved by ⅓ or more of the original value), and ‘poor-responders’ (where their improvement was less than ⅓). It is thought that a variety of factors contribute to resistance and poor response to steroids.

Firstly, where a pathway that cannot be controlled by steroids is involved in pathogenesis, no therapeutic effect from the use of steroids can be expected. Steroid drugs are not applicable in such cases, and hence should not be used. Steroid responsiveness really becomes an issue when an otherwise effective steroid is rendered ineffective as a result of a patient's diathesis.

Patients who are poor steroid responders should be treated with something other than steroids. When administering steroids, it is important to manage side effects such as adrenal cortex dysfunction and eyesight-related problems such as cataracts and glaucoma. Side effects such as dermatrophy, steroid purpura and steroid dermatitis can also be observed when steroids are used topically. Patients who are poor steroid responders should not be unnecessarily exposed to these and other steroid side effects. In these cases it is far more preferable to predict steroid responsiveness prior to steroid administration. Furthermore, medical principle is such that a method of treatment deemed to be effective is selected, regardless of any potential steroid side effects. However, currently there is no way of predicting a patient's steroid responsiveness without the actual administration of steroids.

The cause of steroid-resistance has not been fully elucidated. For example, aberration in the post-translational modification of the steroid-targeted “GR”s been indicated as a possible cause of steroid-resistance (Picard, D. Nature 348:166-168, 1990. Reduced levels of hsp90 compromise steroid receptor action in vivo.). Alternatively, it has also been speculated that, where numerous inflammatory transcription factors are associated with inflammation, the steroid's regulatory limit is exceeded, resulting in steroid resistance. It has been also considered that CBP (CREB-binding protein), a transcriptional coactivator, is consumed by the transcriptional activation of other genes, resulting in insufficient transcription of the gene or genes essential for immunosuppression by steroids (Kamei, Y. et al. Cell 85: 403-414, 1996. A CBP integrator complex mediates transcriptional activation and AP-1 inhibition by nuclear receptors). However, none of these reports sufficiently explain the mechanism of poor steroid responsiveness. In order to predict poor response to steroids it is necessary to elucidate its causes.

Elucidation of the causes of poor steroid responsiveness enables not only the prediction thereof, but also the provision of novel therapeutic methods. For example, if the molecule responsible for reduced steroid responsive can be better understood, inhibition of this molecule can enable increased steroid responsiveness and promotion of the therapeutic effect of steroids. Alternatively, if reduced steroid responsiveness is caused by a quantitative shortage of a specific molecule, supplementary administration of that molecule should improve steroid responsiveness.

A variety of methods have been tried for the treatment of allergic diseases, however steroids remain an important choice of therapy. Steroids are currently the only treatment exacting excellent therapeutic effects on a wide variety of disorders. Thus, an effective treatment for poor steroid responsiveness will be a boon to patients who respond poorly to steroids.

In addition, in activated vitamin D₃ treatment of kidney dialysis patients, morbidity caused by the insufficient therapeutic effects of steroids can be cited as a transition to secondary hyperparathyroidism. Activated vitamin D₃ is a steroid typically used in controlling parathyroid function. However, in patients with poor steroid responsiveness, a transition to secondary hyperparathyroidism can be observed.

Thus, elucidation of the cause of changes in steroid responsiveness is highly significant.

### DISCLOSURE OF THE INVENTION

An objective of the present invention is to provide genes that serve as markers for steroid responsiveness. Furthermore, another objective of the present invention is to provide a method for testing steroid responsiveness and a method of screening for compounds that elevate steroid responsiveness based on the markers.

The present inventors considered that elucidation of genes associated with steroid responsiveness would be useful for diagnosis and treatment of steroid responsiveness. Therefore, the inventors searched for genes whose expression levels differed between patients who responded to steroid treatment and those who only poorly respond thereto. The use of DNA chips is advantageous to observe differences in expression levels of numerous genes among cells under a specific condition. To search for target genes among a wide range of genes, the present inventors used a DNA chip that enables analysis of approximately 5,600 different genes. Furthermore, to discover specific genes whose expression level changes in association with steroid responsiveness and poor responsiveness of subjects, the inventors selected genes with a change in the expression level of 3-fold or more between responsive and poorly responsive subjects.

Next, the expression level of the genes obtained was analyzed in a plurality of atopic dermatitis patients. As a result, the inventors succeeded in isolating genes, RING6 and HLA-DMB, whose expression level was significantly reduced in patients with steroid responsiveness as compared to patients poorly responding to steroid therapy. Furthermore, the inventors found that steroid responsiveness can be tested and compounds to raise steroid responsiveness can be screened using this gene as a marker and completed this invention. Specifically, the present invention relates to a method for testing steroid responsiveness as well as a method of screening for a compound to raise steroid responsiveness as described below:

[1] a method for testing steroid responsiveness, comprising the steps of:

a) measuring the expression level of the RING6 gene or HLA-DMB gene in a biological sample of a test subject; and

b) comparing the measured expression level to that of the same gene in a biological sample taken from either a normal healthy subject or poor steroid responsive subject;

[2] the method according to [1], wherein the steroid responsiveness of an allergic disease is tested;

[3] the method according to [2], wherein the allergic disease is atopic dermatitis;

[4] the method according to [1], wherein the expression level of the gene is measured by PCR of cDNA;

[5] the method according to [1], wherein the expression level of the gene is measured by detecting the protein encoded by the gene;

[6] a reagent for testing steroid responsiveness, said reagent comprising an oligonucleotide having a nucleotide sequence complementary to a polynucleotide comprising the nucleotide sequence of the RING6 gene or HLA-DMB gene or to the complementary strand thereof, which oligonucleotide has a length of at least 15 nucleotides;

[7] a reagent for testing steroid responsiveness, said reagent comprising an antibody recognizing a peptide containing the amino acid sequence of the RING6 protein or HLA-DMB protein;

[8] a method of screening for a compound that elevates steroid responsiveness, comprising the steps of:

(1) contacting a candidate compound with a cell that expresses a gene selected from the group consisting of the RING6 gene, the HLA-DMB gene and genes functionally equivalent thereto;

(2) measuring the expression level of the gene; and

(3) selecting the compound that reduces the expression level of the gene as compared to the expression level associated with a control cell that has not been contacted with the candidate compound;

[9] the method according to [8], wherein the cell is a mononuclear cell line;

[10] a method of screening for a compound that elevates steroid responsiveness, comprising the steps of:

(1) administering a candidate compound to a test animal;

(2) measuring the expression intensity in a biological sample from the test animal of a gene selected from the group consisting of the RING6 gene, the HLA-DMB gene and genes functionally equivalent thereto; and

(3) selecting the compound that reduces the expression level of the gene as compared to the expression level associated with a control animal not administered the candidate compound;

[11] a method of screening for a compound that elevates steroid responsiveness, comprising the steps of:

(1) contacting a candidate compound with a cell transfected with a vector comprising a transcriptional regulatory region of a gene selected from the group consisting of the RING6 gene; the HLA-DMB gene and genes functionally equivalent thereto, and a reporter gene that is expressed under the control of the transcriptional regulatory region;

(2) measuring the activity of the reporter gene; and

(3) selecting the compound that reduces the expression level of the gene as compared to the expression level associated with a control cell that has not been contacted with the candidate compound;

[12] a method of screening for a compound that elevates steroid responsiveness, comprising the steps of:

(1) contacting a candidate compound with a protein selected from the group consisting of the RING6 protein, the HLA-DMB protein and proteins functionally equivalent thereto;

(2) measuring the activity of the protein; and

(3) selecting the compound that reduces the activity of the protein compared to the activity associated with a control protein that has not been contacted with the candidate compound;

[13] a pharmaceutical that elevates steroid responsiveness, which comprises as an effective ingredient a compound obtained by the method according to any one of [8], [10], [11] and [12];

[14] a pharmaceutical that elevates steroid responsiveness, which comprises as the primary active ingredient an anti-sense DNA against the RING6 gene, the HLA-DMB gene or a portion thereof. [15] a pharmaceutical to elevate steroid responsiveness, which comprises as the primary active ingredient an antibody recognizing a peptide comprising an amino acid sequence of the RING6 protein or the HLA-DMB protein;

[16] a therapeutic agent for poor steroid responsive disorders comprising the pharmaceutical according to any one of [13], [14] and [15] in combination with a steroid drug;

[17] a kit for screening a candidate compound for a therapeutic agent for an allergic disease, said kit comprising an oligonucleotide containing at least 15 nucleotides, wherein the oligonucleotide is complementary to a polynucleotide comprising the nucleotide sequence of the RING6 gene, the HLA-DMB gene or the complementary strand thereof, and a cell expressing the RING6 gene or HLA-DMB gene;

[18] a kit for screening a candidate compound for a therapeutic agent for an allergic disease, said kit comprising an antibody recognizing a peptide containing the amino acid sequence of the RING6 protein or HLA-DMB protein, and a cell expressing the RING6 gene or HLA-DMB gene; and

[19] the use of a transgenic non-human vertebrate in which the expression intensity of a gene selected from the group consisting of the RING6 gene, the HLA-DMB gene, and genes functionally equivalent thereto in mononuclear cells is regulated as a steroid responsiveness-regulated model.

The present invention also relates to a method for improving steroid responsiveness comprising the step of administering a compound that can be obtained by the screening method according to any one of the aforementioned [8], [10], [11] and [12]. The present invention further relates to the use of the compounds which can be obtained by the screening method according to any one of the above described [8], [10], [11] and [12] in the preparation of pharmaceuticals to raise steroid responsiveness. Furthermore, the present invention relates to a method for improving steroid responsiveness comprising the step of administering the following agent (a) or (b):

(a) An anti-sense DNA against the RING6 gene or HLA-DMB gene or a portion thereof.

(b) An antibody recognizing a peptide comprising an amino acid sequence of the RING6 protein or HLA-DMB protein.

Moreover, this invention relates to the use of the agent (a) or (b) in the preparation of pharmaceuticals to raise steroid responsiveness.

The RING6 gene and HLA-DMB gene are genes whose existence had been demonstrated. First, the RING6 gene has been reported as an HLA class 11-like gene and is a member of the immunoglobulin family (Kelly, A. P., Monaco, J. J., Cho, S. G., Trowsdale, J., Nature, 353: 571-3, 1991, “A new human HLA class II-related locus, DM.”). Next, HLA-DMB is a gene encoding the DM-locus type B antigen of the human leukocyte antigen. Although the DMA*0103 allele has been found in atopic dermatitis patients, the relationship between DMB and allergic disorders is unknown (Kuwata, S., Yanagisawa, M., Nakagawa, H., Saeki, H., Etoh, T., Miyamoto, M., Juji, T., J. Allergy Clin. Immunol., 98 (6 Pt 2): S192-200, 1996 December, “HLA-DM gene polymorphisms in atopic dermatitis.”). The relationship between the RING6 and HLA-DMB genes and steroid responsiveness also remains unknown. Furthermore, to date, there has been no report of the involvement of RING6 protein and HLA-DMB protein encoded by these genes with steroid responsiveness.

The relationship between these genes and various diseases that have been identified so far, may be found by, for example, searching the OMIM. The OMIM code numbers of RING6 and HLA-DMB genes are 142855 (RING6) and 142856 (HLA-DMB), respectively. Kelly et al. identified the two genes RING6 and RING7 as a new class II immunoglobulin gene family positioned between the HLA-DMA and DOB genes. The RING6 and RING7 genes are presumed to code for the α and β chains of a protein associated with a hitherto unknown class II family. On the other hand, HLA-DMA and HLA-DMB constitute an important functional heterodimer subunit in the class II antigen-presenting pathway. From these facts, RING6 as well as HLA-DMB are likely to be involved in an important reaction of the antigen-presenting system. However, the involvement of either the RING6 gene or the HIA-DMB gene in steroid responsiveness has not yet been demonstrated.

Herein, “steroid responsiveness” refers to the magnitude of the therapeutic effect of a steroid on allergic reactions or inflammatory symptoms that is achieved following its administration. Steroid responsiveness is not only assessed for allergic disorders but also for all kind of diseases for which a steroid treatment is considered effective. Patients whose symptoms ameliorate by steroid administration are designated as steroid-responsive. In contrast, if no therapeutic effect by a steroid is achieved, the subject is referred to as “steroid-resistant”; likewise, those who exhibit only a slight effect are referred to as “poorly steroid responsive”.

The steroid efficacy on allergic disorders can be quantitatively assessed by comparing a diagnostic marker of an allergic symptom. For example, for atopic dermatitis, a typical allergic disorder, the atopic dermatitis/clinical score system has been known (Leicester system, Sowden, J. M. et al., Lancet, 338: 137-40, 1991, “Double-blind controlled crossover study of cyclosporin in adults with severe refractory atopic dermatitis.”). According to the method, the symptoms of dermatitis are numerically expressed based on the progress and developmental location of dermatitis. In addition, the number of peripheral blood eosinophils can be used as a marker of symptoms of allergic disorders. The therapeutic effects of a steroid can be assessed by comparing these markers before and after the administration of the steroid.

In atopic dermatitis, using the clinical score (the modified Leicester score) of the responsiveness to steroid ointment treatment, patients whose score value is improved by ⅓ or more after two weeks from the initiation of the treatment are categorized as “responders”, and patients with an improvement less than ⅓ are categorized as “poor-responders”. For disorders other than atopic dermatitis, patients can be ranked according to their steroid-responsiveness using an assessment scale of therapeutic effect adapted for each disorder.

Herein, the term “allergic disease” is a general term for diseases in which an allergic reaction is involved. More specifically, it is defined as a disease in which an allergen is identified, a strong correlation between the exposure to the allergen and the onset of the pathological change is demonstrated, and the pathological change is proven to have an immunological mechanism. Herein, an immunological mechanism means that immune responses by the leukocytes are induced by the stimulation of the allergen. Examples of allergens include mite antigen and pollen antigen.

Representative allergic diseases include atopic dermatitis, allergic rhinitis, pollen allergy and insect allergy. Allergic diathesis is a genetic factor that is inherited from allergic parents to their children. Familial allergic diseases are also called atopic diseases, and the causative factor that is inherited is the atopic diathesis. The term “asthma” is a general term for atopic diseases with respiratory symptoms among atopic diseases.

A method for testing steroid responsiveness according to the present invention includes the steps of (1) measuring the expression level of the RING6 gene or HLA-DMB gene in a biological sample of a subject, and (2) comparing the measured value with that of a normal healthy subject or poorly steroid-responsive subject. As a result of comparison between the two values, when the expression level of said gene in the subject is significantly reduced compared to that in the normal healthy subject or poor steroid-responder, the subject is judged to be a responder to steroids. Herein, the RING6 gene and HLA-DMB gene serve as markers for steroid responsiveness and, accordingly, are simply referred to as “marker genes”. In the context of the present invention, the terms “RING6 gene” and “HLA-DMB gene” encompasses homologues not only from human but also from other species. Therefore, a marker gene for species other than human, unless otherwise indicated, refers to either an intrinsic RING6 gene or HLA-DMB gene homologue of that particular species or an extraneous RING6 gene or HLA-DMB gene transformed into the body of the particular species.

In this invention, a homologue of the human RING6 gene or HLA-DMB gene refers to a gene derived from species other than human and which hybridizes under stringent conditions to the human RING6 gene or HLA-DMB gene used as a probe. Stringent conditions generally include conditions such as hybridization in 4×SSC at 65° C. followed by washing with 0.1×SSC at 65° C. for 1 h. Temperature conditions for hybridization and washing that greatly influence stringency can be adjusted according to the melting temperature (Tm). The Tm changes with the ratio of constitutive nucleotides in the hybridizing base pairs and the composition of hybridization solution (concentrations of salts, formamide and sodium dodecyl sulfate). Therefore, considering these conditions, those skilled in the art can empirically select appropriate conditions to achieve a stringency equal to the condition described above.

Herein, the expression level of a marker gene includes transcription of the gene to mRNA as well as translation into protein. Therefore, the method for testing steroid responsiveness according to the present invention is performed based on the comparison of the expression intensity of mRNA corresponding to the aforementioned marker gene or the expression level of a protein encoded by the gene.

For comparing the expression levels, usually a standard value is set based on the expression level of the above-described marker gene in a steroid responder group. Based on this standard value, a permissible range is set, for example, at ±2 S.D. Methods for setting the standard value and permissible range based on the measured values of the marker gene are well known in the art. When the expression level of the marker gene in a subject is in the permissible range, the subject is predicted to be a steroid responder. When that is greater than the permissible range, the subject is predicted to be a poor responder.

Measurement of the expression level of the marker gene in the testing for steroid responsiveness according to the present invention can be performed according to gene analytical methods known in the art. More specifically, for example, the hybridization technique using a nucleic acid hybridizing to the marker gene as a probe, and gene amplification technique using a DNA hybridizing to the gene of this invention as a primer can be utilized for the measurement.

Probes and primers used in the testing according to this invention can be designed based on the nucleotide sequence of the above-described marker genes. The nucleotide sequence of the marker gene and amino acid sequence encoded by the gene are known. GenBank accession Nos. for the nucleotide sequences of the marker genes are X62744 (human RING6) and U15085 (HLA-DMB). The nucleotide sequence of RING6 gene is also set forth in SEQ ID NO: 14, and the amino acid sequence encoded by the nucleotide sequence in SEQ ID NO: 15. The nucleotide sequence of HLA-DMB gene is set forth in SEQ ID NO: 16, and the amino acid sequence encoded by the nucleotide sequence in SEQ ID NO: 17.

Furthermore, generally, genes of higher animals are, with high frequency, accompanied by polymorphism. Moreover, many molecules exist for which isoforms, consisting of different amino acid sequences, are produced during the splicing process. Genes containing mutations in the nucleotide sequence due to polymorphisms or isoforms are also included as marker gene of the present invention, so long as they have a similar activity to the above-described marker gene and are associated with steroid responsiveness.

As a primer or probe for the test according to the present invention, a polynucleotide of at least 15 nucleotides and that is complementary to the polynucleotide comprising the nucleotide sequence of the marker gene or the complementary strand thereof can be utilized. Herein, the term “complementary strand” means one strand of a double stranded DNA composed of A:T (U for RNA) and G:C base pairs to the other strand. In addition, “complementary” means not only those completely complementary to a region of at least 15 continuous nucleotides, but also having a homology of at least 70%, preferably at least 80%, more preferably 90%, and even more preferably 95% or higher. The degree of homology between nucleotide sequences can be determined by the algorithm such as BLAST.

Such polynucleotides are useful as probes to detect the marker gene, or as primers to amplify the marker gene. When used as a primer, those polynucleotides comprise usually 15 bp to 100 bp, preferably 15 bp to 35 bp of nucleotides. When used as a probe, DNAs comprising the whole sequence of the marker gene, or a partial sequence thereof (or its complementary strand) that contains at least 15-bp nucleotides can be used. When used as a primer, the 3′ region thereof must be complementary to the marker gene, while restriction enzyme-recognition sequences or tags may be linked to the 5′ site.

The “polynucleotides” of the present invention may be either DNA or RNA. These polynucleotides may be either synthetic or naturally occurring. Herein, the term “oligonucleotide” means a polynucleotide with relatively low degree of polymerization. Oligonucleotides are included in polynucleotides. In addition, DNA used as a probe for hybridization is usually labeled. Examples of labeling methods include those as described below:

nick translation labeling using DNA polymerase I;

end labeling using polynucleotide kinase;

fill-in end labeling using Klenow fragment (Berger, S L, Kimmel, A R. (1987) Guide to Molecular Cloning Techniques, Method in Enzymology, Academic Press; Hames, B D, Higgins, S J (1985) Genes Probes: A Practical Approach. IRL Press; Sambrook, J, Fritsch, E F, Maniatis, T. (1989) Molecular Cloning: a Laboratory Manual, 2nd Edn. Cold Spring Harbor Laboratory Press);

transcription labeling using RNA polymerase (Melton, D A, Krieg, P A, Rebagkiati, M R, Maniatis, T, Zinn, K, Green, M R. Nucleic Acid Res., 12: 7035-7056, 1984); and

non-isotopic labeling of DNA by incorporating modified nucleotides (Kricka, L J. (1992) Nonisotopic DNA Probing Techniques. Academic Press).

For testing steroid responsiveness using hybridization techniques, for example, Northern hybridization, dot blot hybridization, or DNA chip technique may be used. Furthermore, gene amplification techniques, such as RT-PCR method may be used. By using the PCR amplification monitoring method during the gene amplification step in RT-PCR, one can achieve a more quantitative analysis for the gene expression in the present invention.

In the PCR gene amplification monitoring method, the detection target (DNA or reverse transcript of RNA) is hybridized to probes that are dual-labeled at both ends with different fluorescent dyes whose fluorescence cancel each other out. When the PCR proceeds and Taq polymerase degrades the probe with its 5′-3′ exonuclease activity, the two fluorescent dyes become distant from each other and the fluorescence becomes to be detected. The fluorescence is detected in real time. By simultaneously measuring a standard sample in which the copy number of the target is known, it is possible to determine the copy number of the target in the subject sample with the cycle number where PCR amplification is linear (Holland, P. M. et al., Proc. Natl. Acad. Sci. USA 88: 7276-7280, 1991; Livak, K. J. et al., PCR Methods and Applications 4(6): 357-362, 1995; Heid, C. A. et al., Genome Research 6: 986-994, 1996; Gibson, E. M. U. et al., Genome Research 6: 995-1001, 1996). For the PCR amplification monitoring method, for example, ABI PRISM7700 (Applied Biosystems) may be used.

The method of testing steroid responsiveness of the present invention can also be carried out by detecting a protein encoded by the marker gene. Hereinafter, a protein encoded by a marker gene is referred to as a marker protein. Such test methods are, for example, those utilizing antibodies binding to a marker protein, including the Western blotting method, the immunoprecipitation method and the ELISA method.

Antibodies that bind to a marker protein used in the detection may be produced by techniques known to those skilled in the art. Antibodies used in the present invention may be polyclonal or monoclonal antibodies (Milstein, C. et al., Nature 305 (5934): 537-40, 1983). For example, polyclonal antibodies against the marker protein may be produced by collecting blood from mammals sensitized with an antigen and separating the serum from this blood using known methods. As polyclonal antibodies, the serum containing polyclonal antibodies may be used. According to needs, a fraction containing polyclonal antibodies can be further isolated from this serum. Alternatively, a monoclonal antibody can be obtained by isolating immune cells from mammals sensitized with an antigen; fusing these cells with myeloma cells and such; cloning hybridomas thus obtained; and collecting the antibody from the culture as the monoclonal antibody.

To detect the marker protein, these antibodies may be appropriately labeled. Alternatively, instead of labeling the antibodies, a substance that specifically binds to antibodies, for example, protein A or protein G, may be labeled to arrange for indirect detection of the proteins. More specifically, one example of an indirect detection method is ELISA.

A protein or partial peptides thereof that is used as an antigen may be obtained, for example, by inserting a gene or portion thereof into an expression vector, introducing it into an appropriate host cell to produce a transformant, culturing the transformant to express the recombinant protein, and purifying the expressed recombinant protein from the culture or the culture supernatant. Alternatively, oligopeptides consisting of the amino acid sequence encoded by the gene or partial amino acid sequences of the amino acid sequence encoded by the full-length cDNA are chemically synthesized to be used as the antigen.

Furthermore, according to the present invention, the testing for steroid responsiveness can be conducted using not only the expression level of the marker gene but also the activity of the marker protein in a biological sample as a marker. The activity of the marker protein refers to the biological activity inherent in each protein.

In the testing method of this invention, biological samples of subjects are usually used as the test specimen. Although, blood, sputum, tunica mucosa nasi secretion and the like may be used as the biological sample, it is preferable to use peripheral blood mononuclear cells. The method of collecting mononuclear cells from peripheral blood and such is known in the art. Mononuclear cells isolated, in particular, from peripheral blood are referred to as peripheral blood mononuclear cell (PBMC). Mononuclear cells can be easily collected from heparinized blood, for example, by the specific gravity centrifugation method. Mononuclear cells are a cell population containing monocytes and lymphocytes. The use of mononuclear cells present in a large quantity in peripheral blood facilitates the collection of test samples. Thus, a simple bedside test becomes possible. Lysate prepared by fragmenting the isolated mononuclear cells can be used as a specimen for immunological measurement of the above-described protein. Alternatively, mRNA extracted from this lysate may be used as a specimen for the measurement of mRNA corresponding to the aforementioned marker gene. The extraction of lysate and mRNA from mononuclear cells can be conveniently carried out using commercial kits. Moreover, when the marker protein is secreted into the blood stream, the amount of this target protein contained in a humor sample, such as blood and serum of subjects, may be measured to enable comparison of the expression levels of the gene encoding said protein. According to needs, the aforementioned specimens can be used in the method of this invention after being diluted with a buffer and the like.

In the case of measuring mRNA, in the present invention, the measured value of the RING6 gene or HLA-DMB gene expression level can be corrected by known methods. The correction enables comparison of changes in the expression levels of the gene in cells. According to this invention, based on the measured value of the expression level of a gene (for example, a housekeeping gene) whose expression level in each cell in the above-described biological samples does not widely fluctuate, the measured values of the expression levels of the RING6 gene or HLA-DMB gene are corrected. Examples of genes whose expression levels do not widely fluctuate include those encoding β-actin and GAPDH.

Tests for steroid responsiveness in the present invention include the following. Specifically, when steroid treatment is applied to a patient showing atopic dermatitis symptoms, steroid responsiveness of the patient can be predicted based on the present invention prior to the administration of steroids. More specifically, the decrease in the expression level of the marker gene in a patient indicates a high possibility that the patient is a responder to steroid, and steroid therapy may be effective for such a patient.

Steroid administration is accompanied by the risk of side effects as described above. Furthermore, prediction of therapeutic effects prior to the initiation of treatment leads to immediate relief of patient from agony to improve his/her quality of life (QOL). Therefore, the testing method of the present invention provides extremely important information on the selection of therapeutic plans for allergic diseases.

Alternatively, a gene whose expression level changes in response to steroid can be expected to be useful as a marker for the decrease of type 1 helper T cells (Th1 cells). The decrease of Th1 cell function in comparison to the type 2 helper T cells (Th2 cells) is considered as one of the causes of allergic diseases. According to this concept, allergic symptoms are caused because of relative enhancement of the function of Th2 cells inducing IgE antibody production to Th1 cells. The increase in the number of Th2 cells and decrease of Th1 cells may be the cause of the relative decrease of the function of Th1.

Patients with atopic dermatitis (AD) with decreased IFN-γ productivity have been reported to have increased levels of IgE antibody specific to Candida (Kimura, M., Tsuruta, S., Yoshida, T., Int. Arch. Allergy Immunol. 122: 195, 2000, “IFN-gamma plays a dominant role in upregulation of Candida-specific IgE synthesis in patients with atopic dermatitis.”). IFN-γ is a typical Th1 cytokine. Thus, patients with AD due to the decrease in Th1 cells have decreased resistance to fungi and viruses and thus resident Candida is likely to be increased. As a result, the raised IgE level against Candida may explain the increased type I allergic reactions.

Such patients are predicted to show further aggravated inflammatory symptoms due to infections with Candida, etc. and allergy. Furthermore, administration of steroids to such patients is likely to lead to a further decrease in Th1 cell function, which is already reduced, due to the suppressing effect of steroids. Thus, the decrease in Th1 cells may be one of the causes of poor steroid responsiveness. Therefore, genes whose expression level changes in response to steroid responsiveness are expected to be useful as markers of Th1 cell decrease as well. Patients having allergic diseases caused by the decrease of Th1 cells not only are poor responders to steroids, but steroid treatments may also involve the risk of causing exacerbation of symptoms in such patients. Therefore, genes that serve as markers of the balance between Th1 and Th2 cells prior to steroid administration are useful.

Alternatively, the testing method according to the present invention can be utilized as a marker of the effectiveness of a steroid treatment after it is initiated. For example, when the expression level of the marker gene fails to decrease even after commencing steroid treatment, the subject is presumed to be a poor responder to the steroid used. Accordingly, alternative steroid therapies should be considered. Furthermore, the test method of the present invention may be performed on a patient showing a clearly visible steroid therapy effect at the time of treatment initiation. When a decrease in the expression level of a marker gene is observed, the patient is predicted to be a steroid responder. No problems arise so long as the steroid is therapeutically effective on such a patient. However, when the expected therapeutic effect fails to present, it seems worthwhile to try other treatments besides steroid therapy.

Moreover, the present invention also relates to the use of transgenic, non-human vertebrates as model animals of steroid responsiveness, wherein the expression level of a marker gene in mononuclear cells has been manipulated or adjusted to reflect a desired degree of steroid responsiveness. In the context of the present invention, the regulation of expression level refers to the elevation or reduction of the expression of a marker gene. In the present invention, the elevation of marker gene expression leads to the reduction of steroid responsiveness. That is, the present invention enables the production of model animals with a poor steroid responsiveness. In contrast, it is possible to produce a state of elevated steroid responsiveness by reducing the expression level of a marker gene, that is, to obtain steroid responsive model animals.

Allergic disease model animals having a poor steroid responsiveness may be used to elucidate in vivo changes in poor steroid-responsive atopic dermatitis. Furthermore, allergic disease model animals of the present invention having a poor steroid responsiveness may be used to evaluate therapeutic methods for poor steroid-responsive allergic atopic dermatitis. Moreover, the poor steroid-responsive animals of the present invention may be used to screen for compounds that suppress the expression and activity of marker genes.

Alternatively, steroid-responsive model animals of the present invention can be used to screen for compounds with a steroid-like activity. Since a steroid-responsive model animal obviously responds to steroids, a compound that causes a similar change in the marker level in this animal as that observed at the time of steroid administration can be expected to have a steroid-like activity.

The decrease in the expression levels of the aforementioned marker gene in mononuclear cells in patients with steroid-responsive allergic disorders is demonstrated by the present invention. Therefore, animals wherein the expression levels of the marker gene in mononuclear cells are artificially enhanced can be used as model animals for poorly steroid-responsive diseases.

Herein, the increase (or decrease) in the expression level in mononuclear cells includes the increase (or decrease) in the expression level of the marker gene in the whole blood cells. Specifically, the increase (or decrease) in the expression level of the above-described marker gene includes not only that merely in the mononuclear cell but also that in the whole blood cells and systemic increase (or decrease) of the marker gene.

In the present invention, a functionally equivalent gene refers to a gene encoding a protein having a similar activity to that demonstrated in the protein encoded by the marker gene. A typical functionally equivalent gene includes a counterpart of a marker gene inherent in the species of the transgenic animal.

The model animals of poorly steroid responsive diseases according to the present invention are particularly useful as model animals of poorly steroid responsive allergic diseases.

A gene whose expression level is reduced in a steroid-responsive allergic disease is likely to be a gene that suppresses responsiveness to steroid drugs. In other words, poor steroid responsiveness is likely to be a state in which elevated expression of a marker gene prevents the transmission of the stimulation of a steroid drug. That is, a gene whose expression level is reduced in a steroid-responsive allergic disease compared to a poorly steroid-responsive allergic disease is likely to play an important role in the suppression of steroid responsiveness. Therefore, in the steroid therapy for allergies, drugs that suppress the expression of marker genes or inhibit the activity thereof can be expected to remove the intrinsic cause of poor steroid responsiveness. Furthermore, effective steroid therapy can be achieved by suppressing the activity of proteins encoded by these marker genes. To suppress gene expression, decoy nucleic acid drugs and anti-sense drugs can be utilized. It is also possible to suppress the protein activity using, for example, an antibody that inhibits the protein activity or a compound that specifically binds to the active site of the protein.

As described herein, a gene whose expression level is lowered in mononuclear cells of steroid responsive allergic disease patients is highly significant. Therefore, a transgenic animal of the present invention having controlled steroid responsiveness finds significant utility when evaluating the role of the gene and the efficacy of drugs targeting the gene.

Alternatively, the above-described transgenic animals can be used to screen for non-steroidal drugs useful for the treatment of allergic diseases. That is, compounds that cause changes similar to those observed by the administration of steroids may be identified using the aforementioned transgenic animals, which, in turn, enables the selection of compounds expected to have a steroid-like therapeutic effect yet a reaction mechanism different from that of steroid. Examples of such “similar changes” observed by administering steroids include, but are not limited to, expression changes of Th1 cytokines and the like.

Moreover, the poorly steroid-responsive model animal according to the present invention is useful in the elucidation of steroid response mechanisms and further in testing safety of screened compounds. The model animals for poorly steroid-responsive disorders according to the present invention are particularly useful as models for poorly steroid-responsive allergic diseases.

Herein, the phrase “increase in the expression level” refers to a state wherein the transcription of the marker gene inherent in the host, and translation of the gene to protein are enhanced. Alternatively, it may refer to a state with inhibited degradation of proteins or translation products of the gene. The expression level of a gene can be confirmed, for example, by quantitative PCR as shown in Examples. Moreover, the activity of a protein, a translational product, can be confirmed by a comparison to that in the normal state.

Typical transgenic animals include those to which a marker gene has been introduced. Other examples include animals having a mutation introduced into the coding region of a marker gene so as to elevate the activity thereof or those having a modified amino acid sequence such that the gene product is a hardly degradable sequence. Examples of amino acid sequence mutations include the substitution, deletion, insertion, or addition of amino acid residues. Furthermore, the expression of a marker gene of this invention can be regulated by mutating the transcriptional regulatory region of the gene.

On the other hand, a transgenic animal which has been transduced with an anti-sense DNA against a marker gene (including the homologous gene in a test animal), DNA coding for ribozyme, or DNA functioning as a decoy nucleic acid, or the like, can be used as a transgenic animal in which the function of a marker gene of the present invention has been reduced. Furthermore, animals in which a mutation has been introduced into the coding region of a marker gene so as to suppress the activity thereof, or those having a modified amino acid sequence that results in a gene product susceptible to degradation may be cited as transgenic animals having a reduced marker gene expression level.

Methods for obtaining transgenic animals with a particular target gene are known in the art. Specifically, a transgenic animal can be obtained by a method wherein the target gene and ovum are mixed and treated with calcium phosphate; a method where the target gene is introduced directly into the nucleus of oocyte in pronuclei with a micropipette under a phase contrast microscope (microinjection method, U.S. Pat. No. 4,873,191); or a method where embryonic stem cells (ES cells) are used. Furthermore, new developments include a method for infecting ovum with a gene-inserted retrovirus vector, a sperm vector method for transducing a gene into ovum via sperm, and such. The sperm vector method is a gene recombination technique for introducing a foreign gene by fertilizing ovum with sperm after a foreign gene has been incorporated into sperm by the adhesion or electroporation method, and so on (M. Lavitranoet et al., Cell, 57: 717, 1989).

Transgenic animals used as regulated steroid responsive model animals of the present invention can be produced using all the vertebrates except for humans. More specifically, transgenic animals having various transgenes and showing modified gene expression levels are produced using vertebrates such as mice, rats, rabbits, miniature pigs, goats, sheep, monkeys and cattle.

Furthermore, the present invention relates to a method of screening for a compound to raise steroid responsiveness in a subject. According to this invention, the expression level of a marker gene is significantly lowered in mononuclear cells of patients with steroid-responsive allergic diseases. Therefore, compounds that enhance steroid responsiveness can be obtained by selecting compounds that reduce the expression level of the marker gene. The screening method of this invention is particularly preferable for screening for candidate compounds useful in improving steroid responsiveness in patients suffering from poorly steroid-responsive allergic diseases. “Compounds that reduce the expression level of a gene” as used herein means those having inhibitory functions on any of the steps of transcription and translation of the gene as well as the expression of the activity of the translated protein.

The method of screening for a compound to raise steroid responsiveness of the present invention can be performed either in vivo or in vitro. This screening can be conducted, for example, according to the following steps:

(1) administering a candidate compound to a test animal;

(2) measuring the expression level of the above-described marker gene in a biological specimen of the test animal; and

(3) selecting a compound that reduces the expression level of the marker gene as compared to that in the control administered with no candidate compound.

According to the screening method of the present invention, a gene selected from the group consisting of the RING6 gene, the HLA-DMB gene and genes functionally equivalent thereto can be used as marker genes. The phrase “functionally equivalent” herein refers to a gene encoding a protein having a similar activity to that demonstrated in the protein encoded by the marker gene. A typical functionally equivalent gene includes a counterpart of an indictor gene inherent in the particular animal species of the test animal.

As a test animal in the screening method of the present invention, for example, a poorly steroid-responsive transgenic animal in which a human marker gene has been forcibly expressed can be used. If a promoter whose transcriptional regulating activity is controlled by a substance such as an appropriate drug is used, the expression level of an exogenous marker gene in the transgenic animal can be regulated by administering the substance.

Thus, the effect of a drug candidate compound on the expression level of the marker gene can be detected by administering the compound to a marker gene forced expression model animal and monitoring its action on the expression of the marker gene in a biological specimen from the model animal. The changes in the expression level of the marker gene in the biological specimen of the test animal can be monitored by a similar technique to the above-described test method of this invention. Furthermore, the screening for drug candidate compounds can be achieved by selecting drug candidate compounds that reduce the expression level of the marker gene based on this detection result.

More specifically, the screening according to the present invention can be carried out by collecting a biological specimen from a test animal to compare the expression level of the aforementioned marker gene to that in a specimen taken from a control animal treated with no candidate compound. The biological specimens that can be used include lymphocytes and hepatocytes. Preferable biological specimens in the screening method according to this invention are peripheral blood mononuclear cells. Methods for collecting and preparing such biological specimens are known in the art.

The screening enables selection of drugs associated with the expression of the marker gene in various modes of actions. Specifically, drug candidate compounds having, for example, following actions can be discovered:

(1) suppression of the signal transduction pathway that induces expression of the marker gene;

(2) reduction of the transcriptional activity of the marker gene;

(3) destabilization of the transcripts of the marker gene or enhancement of decomposition of the transcript, and so on.

Moreover, an in vitro screening method includes, for example, the steps of contacting a candidate compound with a cell that expresses a marker gene and selecting the compound that reduces the expression level of the gene. More particularly, the screening can be conducted, for example, according to the steps as described below:

(1) contacting a cell expressing the marker gene with a candidate compound;

(2) measuring the expression level of the marker gene; and

(3) selecting a compound that reduces the expression level of the marker gene as compared to that in control cells that have not been contacted with the candidate compound.

In this invention, cells expressing the marker gene can be obtained by inserting the marker gene into an appropriate expression vector and then transfecting suitable host cells with the vector. Any vectors and host cells may be used so long as they are capable of expressing the gene of this invention. Examples of host cells in the host-vector system are Escherichia coli cells, yeast cells, insect cells and animal cells, and available vectors usable for each can be selected.

Vectors may be transfected into the host by biological methods, physical methods, chemical methods, and the like. Exemplary biological methods include methods using virus vectors; methods using specific receptors; and the cell-fusion method (HVJ (Sendai virus) method, the polyethylene glycol (PEG) method, the electric cell fusion method and microcell fusion method (chromosome transfer)). Exemplary physical methods include the microinjection method, the electroporation method and the method using gene particle gun. The chemical methods are exemplified by the calcium phosphate precipitation method, the liposome method, the DEAE-dextran method, the protoplast method, the erythrocyte ghost method, the erythrocyte membrane ghost method and the microcapsule method.

In the screening method of the present invention, peripheral blood leucocytes and cell lines derived therefrom can be used as cells expressing a marker gene. Mononuclear cells and immature neutrophils can be mentioned as leucocytes. Among them, lymphoid cell lines are preferable for the screening method of this invention.

According to the screening method of the present invention, first, a candidate compound is added to the above-described cell line. Then, the expression level of the marker gene in the cell line is measured to select a compound that reduces the expression level of the marker gene compared to a control that has not been contacted with the candidate compound.

In the screening method of the present invention, the expression level of the marker gene can be compared not only based on the expression level of the protein encoded by the gene but also by detecting mRNAs corresponding to the gene. To compare the expression level by mRNA, the step of preparing mRNA samples as described above is carried out in place of the step for preparing a protein sample. mRNA and protein can be detected by performing known methods as mentioned above.

Furthermore, based on the disclosure of this invention, transcriptional regulatory regions of a marker gene of this invention can be obtained to construct a reporter assay system. The phrase “reporter assay system” refers to an assay system for screening a transcriptional regulatory factor that acts on a transcriptional regulatory region using the expression level of a reporter gene that is located downstream of the transcriptional regulatory region as a marker.

Specifically, this invention relates to a method of screening for therapeutic agents to raise steroid responsiveness, which comprises the steps of:

(1) contacting a candidate compound with a cell transfected with a vector containing the transcriptional regulatory region of a marker gene and a reporter gene that is expressed under the control of this transcriptional regulatory region;

(2) measuring the activity of the above-described reporter gene; and

(3) selecting a compound that reduces the expression level of the reporter gene compared to that in a control

wherein the marker gene is a gene selected from the group consisting of the RING6 gene or HLA-DMB gene or a gene functionally equivalent thereto.

Examples of transcriptional regulatory regions include promoters and enhancers, as well as the CAAT box, the TATA box and the like which are usually found in a promoter region. Reporter genes such as the chloramphenicol acetyltransferase (CAT) gene, the luciferase gene, growth hormone genes and the like can be utilized in the present invention.

The transcriptional regulatory region of the RING6 gene has been described in literature (Beck, S., Abdulla, S., Alderton, R. P., Glynne, R. J., Gut, I. G., Hosking, L. K., Jackson, A., Kelly, A., Newell, W. R., Sanseau, P., Radley, E., Thorpe, K. L. and Trowsdale, J., J. Mol. Biol., 255 (1): 1-13m, 1996, “Evolutionary dynamics of non-coding sequences within the class II region of the human MHC” (accession; X87344)). The identified transcriptional regulatory region has been mapped on the genome sequence as follows: (GenBank Acc. No. X87344; H. sapiens DMA, DMB, HLA-Z1, IPP2, LMP2, TAP1, LMP7, TAP2, DOB, DQB2 and RING8, 9, 13 and 14 genes.). Of the nucleotide sequence registered as X87344, the parts containing the following respective regions are set forth in SEQ ID NO: 18.

843 to 856: GC signal

1273 to 1282: J-box

1286 to 1304: X-box

1324 to 1333: Y-box

1354 to 1359: CAAT signal

1398 to 1411: GC signal

1467 to 1554: hypothetical exon

(the gene region spanning 1467 to 5873.)

1790 to 1802: promoter (ISRE sequence)

1966 to 2041: alternative exon 1 (hypothetical)

1991 to 2000: promoter (NFκB sequence)

The transcriptional regulatory region of the HLA-DMB gene has also been described in literature, particularly in the above-cited Beck, S. and Radley, E. et al. (Radley, E. et al., J. Biol. Chem., 269 (29): 18834-18838, 1994, “Genomic organization of HLA-DMA and HLA-DMB. Comparison of the gene organization of all six class II families in the human major histocompatibility complex”, accession; X76776). The identified transcriptional regulatory region has been mapped on the genome sequence as follows. Of the nucleotide sequence registered as X76776, parts containing the following sections are set forth in SEQ ID NO: 19. Exon 1 in SEQ ID NO: 19 corresponds to nucleotides 756 to 810, and exon 2 is located downstream of nucleotide 2598. In this case, HLA-DMB is composed of 6 exons.

19 to 28: promoter (NFKB sequence)

73 to 82: promoter (J-box)

119 to 128: promoter (J-box)

134 to 147: promoter (Sp1 sequence)

313 to 322: promoter (J-box)

439 to 448: promoter (J-box)

440 to 458: promoter (X-box)

478 to 487: promoter (Y-box)

513 to 517: CAAT sequence

574 to 588: promoter (Sp1 sequence)

582 to 591: promoter (NFκB sequence)

740 to 749: promoter (J-box)

829 to 838: promoter (NFκB sequence)

Alternatively, a transcriptional regulatory region of the marker gene of the present invention can be obtained as follows. Specifically, first, based on the nucleotide sequence of the marker gene disclosed in this invention, a human genomic DNA library, such as BAC library and YAC library, is screened by a method using PCR or hybridization to obtain a genomic DNA clone containing the sequence of the cDNA. Based on the sequence of the obtained genomic DNA, the transcriptional regulatory region of a cDNA disclosed in this invention is predicted and obtained. The obtained transcriptional regulatory region is cloned upstream of a reporter gene to prepare a reporter construct. The obtained reporter construct is introduced into a cultured cell strain to prepare a transformant for screening. By contacting a candidate compound with this transformant, screening for the compound that controls the expression of the reporter gene can be performed.

As an in vitro screening method according to this invention, a method based on the activity of a marker protein can be utilized. That is, the present invention relates to a method of screening for therapeutic agents that raise steroid responsiveness, which comprises the steps of:

(1) contacting a candidate compound with a protein encoded by a marker gene;

(2) measuring the activity of the protein; and

(3) selecting a compound that reduces the activity of the protein compared to a control, wherein the marker gene is a gene selected from the group consisting of the RING6 gene or HLA-DMB gene and a gene functionally equivalent thereto.

The activities of RING6 and HLA-DMB, the marker proteins of this invention, are already described above. Using the activity as a marker, compounds having the activity to inhibit the activity of the marker protein can be screened. The compounds that can be obtained by the method, suppress the activity of the RING6 and HLA-DMB. As a result, it is possible to control poorly steroid-responsive allergic diseases through the inhibition of the marker protein whose expression in mononuclear cells is induced.

Test candidate compounds used in these screening methods include, in addition to compound preparation libraries synthesized by combinatorial chemistry, mixtures of multiple compounds such as extracts from animal or plant tissues, or microbial cultures and their purified preparations.

The polynucleotide, antibody, cell line or model animal, which are necessary for the various methods of screening of this invention, can be combined in advance to produce a kit. More specifically, such a kit may comprise, for example, a cell that expresses the marker gene and a reagent for measuring the expression level of the marker gene. As a reagent for measuring the expression level of the marker gene, for example, an oligonucleotide that has at least 15 nucleotides complementary to the polynucleotide comprising the nucleotide sequence of at least one marker gene or to the complementary strand thereof is used. Alternatively, an antibody that recognizes a peptide comprising the amino acid sequence of at least one marker protein may be used as a reagent. These kits may further include a substrate compound used for the detection of the marker, medium and a vessel for cell culturing, positive and negative standard samples, and furthermore, a manual describing how to use the kit.

Compounds selected by the screening methods of this invention are useful as drugs that raise steroid responsiveness. In the context of the present invention, a drug that raises steroid responsiveness can be formulated by including a compound selected by the above-described screening methods as the effective ingredient, and mixing it with physiologically acceptable carrier, excipient, diluent and the like. For improving steroid responsiveness in patients with disorders for whom the administration of steroid drugs has been selected as a therapeutic method, the drug that raises steroid responsiveness of the present invention can be administered orally or parenterally. Disorders for which the drug of this invention is applied include poorly steroid responsive allergic diseases. Alternatively, when the compound to be administered consists of a protein, a therapeutic effect can be achieved by introducing a gene encoding the protein into the living body using techniques of gene therapy. Techniques for treating disorders by introducing, into the living body, a gene encoding a protein with a therapeutic effect and expressing the gene in vivo is known in the art.

Examples of drugs that can suppress the expression of a marker gene of the -present invention include, for example, anti-sense DNA and decoy nucleic acids. Anti-sense DNA can be constructed by arranging a marker gene of the present invention, or a portion thereof, in the opposite direction at the downstream of the promoter. Administration of a vector capable of expressing the anti-sense DNA to a patient enables the inhibition of the expression of the marker gene in cells transformed by the vector. On the other hand, a decoy nucleic acid, or a DNA containing the expression regulatory region of a marker gene, competitively inhibits the action of transcription factors by its transduction into cells. Such therapeutic methods for inhibiting gene expression through the transduction of a specific gene are well known.

Furthermore, compounds that inhibit the activity of proteins (i.e. marker proteins) that are expression products of the marker genes of this invention, are also expected to have the action of enhancing steroid responsiveness. For example, antibodies that recognize the marker proteins of this invention and suppress their activity are useful as pharmaceutical agents for enhancing steroid responsiveness. Methods for preparing antibodies that suppress protein activity are well known. For administration to humans, antibodies may be prepared as chimeric antibodies, humanized antibodies, or human-type antibodies to serve as highly safe pharmaceutical agents.

For oral drugs, any dosage forms, including granules, powders, tablets, capsules, solutions, emulsions and suspensions, may be selected. Examples of injections contemplated herein include subcutaneous, intramuscular and intraperitoneal injections.

Moreover, compounds that can be obtained by the screening methods of this invention, anti-sense DNA against the marker gene and antibodies include those having the activity to improve and raise steroid responsiveness of patients and which thus are useful as drugs. Such drugs can be formulated as therapeutic agents for poorly steroid-responsive diseases by combining them with steroids.

Although the dosage may vary depending on the age, sex, body weight, symptoms of a patient, treatment effects, method for administration, treatment duration, type of active ingredient contained in the drug composition, etc., a range of 0.1 to 500 mg, preferably, 0.5 to 20 mg per dose for an adult can be administered. However, the dose changes according to various conditions, and thus, in some cases, a smaller amount than that mentioned above is sufficient whereas in other cases, a greater amount is required in other cases.

All the literatures for prior arts cited in the present specification are herein incorporated by reference.

### BRIEF DESCRIPTION OF THE DRAWINGS

FIG. 1 represents bar graphs showing the results of the measurements on the RING6 gene expression levels in the steroid responder group, poor steroid responder group and normal healthy individuals. The upper graph shows the measured values (copy/ng RNA) in each subject corrected for the β-actin gene. The lower graph represents the results of statistical analysis among respective groups. Herein, V represents a normal healthy subject, R the steroid responder group, and P the poor steroid responder group. Numerals are the reference numbers of respective subjects.

FIG. 2 represents bar graphs showing the results of the measurements on the RING6 gene expression levels in the steroid responder group, poor steroid responder group and normal healthy individuals. The upper graph shows the measured values (copy/ng RNA) in each subject corrected for the GAPDH gene. The lower graph represents the results of statistical analysis among respective groups. Herein, V, R and P are the same as FIG. 1, respectively. Numerals are the reference numbers of respective subjects.

FIG. 3 represents bar graphs showing the results of the measurements on the HLA-DMB gene expression levels in the steroid responder group, poor steroid responder group and normal healthy individuals. The upper graph shows the measured values (copy/ng RNA) in each subject corrected for the ,-actin gene. The lower graph represents the results of statistical analysis among respective groups. Herein, V, R and P are the same as FIG. 1, respectively. Numerals are the reference numbers of respective subjects.

FIG. 4 represents bar graphs showing the results of the measurements on the HLA-DMB gene expression levels in the steroid responder group, poor steroid responder group and normal healthy individuals. The upper graph shows the measured values (copy/ng RNA) in each subject corrected for the GAPDH gene. The lower graph represents the results of statistical analysis among respective groups. Herein, V, R and P are the same as FIG. 1, respectively. Numerals are the reference numbers of respective subjects.

### BEST MODE FOR CARRYING OUT THE INVENTION

The present invention will be explained in more detail below with reference to examples, but it is not to be construed as being limited thereto.

### EXAMPLE 1

### Selection of Candidate Gene Using DNA Chip

(1) Mononuclear Cells

Heparinized blood samples were withdrawn from 2 normal healthy volunteers (hereinafter referred to as “normal group”), 3 responders to steroid ointment treatment and 3 poor-responders thereto (hereinafter referred to as “steroid responder group” and “poor steroid responder group”, respectively; also both groups collectively referred to as “patient group”). Then the blood samples were subjected to specific gravity centrifugation according to following method for collecting mononuclear cell fractions to culture the fractions.

40-ml of the whole blood (using a heparin anticoagulant at a final concentration of 50 unit/ml) was placed in a centrifuge tube; an equal volume of 3% dextran/0.9% NaCl was added and mixed by gently tumbling the tube several times. The resulting mixture was left standing at room temperature for 30 min. Then, the supernatant (platelet rich plasma) was recovered and centrifuged at 1,200 rpm (revolutions per minute) at room temperature for 5 min. After removing the supernatant, the pellet was suspended in Hank's Balanced Salt Solutions (HBSS, GIBCO BRL) (5 ml), layered on Ficoll-Paque™ PLUS (Amersham Pharmacia Biotech) (5 ml), centrifuged at 1,200 rpm at room temperature for 5 min and further for 30 min raising the rpm to 1,500 at room temperature. The supernatant was removed to recover the intermediate layer. The recovered layer was suspended in PBS and centrifuged at 1,500 rpm at room temperature for 5 min. The supernatant was discarded. The pellet was re-suspended in PBS and centrifuged at 1,500 rpm at room temperature for 5 min. The pellet thus obtained was suspended in RPMI1640 (GIBCO BRL)/10% FCS (SIGMA) (10 ml). 20 μl of the suspension was subjected to cell staining with Trypan Blue Stain 0.4% (GIBCO BRL) to count the cell number. A suspension (1.5×10⁶ cells/ml) in RPMI1640/10% FCS (10 ml) was prepared and cultured at 37° C. in a 5% CO₂ atmosphere for 24 h. Then total RNA was extracted according to following method.

Total RNA was extracted using RNA extraction kit, ISOGEN (Nippon Gene) according to the accompanying direction. The cultured cells were lysed in Isogen (4 M guanidium thiocyanate, 25 mM sodium cyanate, 0.5% Sarcosyl, 0.1 M β-mercaptoethanol, pH 7.0) (3 ml). Suction using a 2.5-ml syringe with a 20G Cathelin needle was repeated 20 to 30 times. CHCl₃ (0.6 ml, ⅕ volume of Isogen) was added to the extract, mixed for 15 sec using a mixer and the mixture was left standing at room temperature for 2 to 3 min. Then, the mixture was centrifuged at 15,000 rpm, 4° C. for 15 min. The supernatant was transferred into a fresh tube, Ethachinmate (Nippon Gene) (3 μl) and isopropanol (1.5 ml, ½ volume to Isogen) were added thereto, mixed by tumbling and the resulting mixture was left standing at room temperature for 10 min. After the mixture was centrifuged at 15,000 rpm, 4° C. for 15 min, 75% ethanol (3 ml, equal volume to Isogen) was added to the precipitate, and the mixture was centrifuged at 15,000 rpm, 4° C. for 5 min. The precipitate was air-dried or vacuum-dried for 2 to 3 min, and RNase-free DW (10 μl) was added to prepare an RNA solution.

(2) Synthesis of cDNA for DNA Chip

Single-stranded cDNA was prepared by reverse-transcription from the total RNA (2 to 5 μg) using a T7-(dT)₂₄ (Amersham Pharmacia Biotech) as a primer and Superscript II Reverse Transcriptase (Life Technologies) according to the method described in Expression Analysis Technical Manual (Affymetrix). The T7-(dT)₂₄ primer consists of the nucleotide sequence of T7 promoter to which (dT)₂₄ is added. T7-(dT)₂₄ primer (SEQ ID NO: 1):

5′-GGCCAGTGAATTGTAATACGACTCACTATAGGGAGGCGG-(dT)₂₄-3′

Then, according to the Expression Analysis Technical Manual, DNA Ligase, DNA polymerase I and RNase H were added to the above-described single-stranded cDNA to synthesize a double-stranded cDNA. The cDNA was purified by phenol-chloroform extraction, passing through Phase Lock Gels and ethanol precipitation.

Furthermore, using BioArray High Yield RNA Transcription Labeling Kit, biotinylated cRNA was synthesized, purified using an RNeasy Spin column (QIAGEN) and then fragmented by heat treatment.

12.5 μg of cRNA was added to a Hybridization Cocktail according to the Expression Analysis Technical Manual. The resulting mixture was added to a DNA microarray, and subjected to hybridization at 45° C. for 16 h. GeneChip^{R} HuGeneFL (Affymetrix) was used as the DNA chip, which is composed of probes consisting of the nucleotide sequences derived from approximately 5600 kinds of human cDNAs and ESTs.

The DNA chip was washed and then stained by adding Streptavidin Phycoerythrin thereto. After washing, an antibody mixture containing normal goat IgG and biotinylated goat anti-streptavidin IgG antibody was added to the microarray. Furthermore, to enhance the fluorescence intensity, the microarray was re-stained by adding Streptavidin Phycoerythrin. After washing, the microarray was set on a scanner and analyzed with GeneChip Software.

(3) DNA Chip Analysis

The expressed fluorescence intensities were measured for data analyses using DNA chip analysis software, Suite. First, all of the chips were subjected to Absolute analysis to measure the gene expression level in each of the used samples.

In the analysis of a single chip data, the fluorescence intensities of the perfect match and mismatch of the probe set were compared to determine positive and negative fractions. The results were classified based on the values of Positive Fraction, Log Avg and Pos/Neg into three groups of Absolute Calls: P (present), A (absent) and M (marginal). Definitions of these terms are described below:

Positive Fraction: ratio of Positive pairs;

Log Avg: logarithmic mean of fluorescence intensity ratios between perfect match and mismatch probe cells; and

Pos/Neg: ratio of Positive pair numbers and Negative pair numbers.

Moreover, Average Difference (Avg Diff), i.e., the mean value of the difference in the fluorescence intensity between perfect match and mismatch probe cells was also calculated.

Next, two data were compared. In the comparative experiment, a chip for standard was determined, and Comparison Analysis was performed using the total gene expression level of the standard chip as a reference standard. Comparison Analysis was performed for one steroid responsive patient against 3 poor steroid responsive patients and the result was used as the standard. Genes whose expression levels in the steroid responsive patient used as the standard are high were limited to genes with a fold change value, one of the calculated values in the software, of −3 or less and at the same time to those satisfying either (i) or (ii) as follows:

(i) genes with a gene expression judgment standard (Absolute call) of P (present) in steroid responsive patients; and

(ii) genes with a gene expression judgment standard (Absolute call) of A (absent) or M (marginal) in poor steroid responsive patients, and with an expression judgment standard M (marginal) in steroid responsive patients. Then, genes with a difference call value of NC (Not change), MD (Marginal Decrease) or D (Decrease) were selected. On the other hand, genes whose expression levels are low were limited to genes with a fold change value of 3 or more, and at the same time satisfying (i) or (ii) as follows:

(i) genes with an Absolute call of P (present) in poor steroid responsive patients; and

(ii) genes with an Absolute calls of A (absent) or M (marginal) in steroid responsive patients, and an expression judgment standard of M (marginal) in poor steroid responsive patients. Then, genes with a difference call value of NC (Not change), MD (Marginal Decrease) or D (Decrease) were selected. Next, according to a graph using scattered plots of Avg Diff values in the log scale, genes plotted near the origin were omitted.

As for genes selected using an analytical software, Suite, genes selected according to the results of 6 different analyses based on two standard patients were chosen among the genes with a high gene expression level in normal healthy subjects.

Response 1 vs. Poor response 1, poor response 2, poor response 3

Response 2 vs. Poor response 1, poor response 2, poor response 3

The classification of genes selected by GeneChip Comparison Analysis showing similar expression changes in the poor steroid responder group by the above-described 6 different combinations are shown in Table 1. Genes with a change of 3-fold or more, or ⅓ or less from the raw data measured values are shown.

TABLE 1

Poor responder group

| | Increase | Decrease |
|---|---|---|
| Responder group | 4 | 2 |

To correlate the results with ABI7700, the expression levels were respectively corrected for the p-actin gene based on Avg Diff values of Absolute analysis to finally select genes showing interesting changes between the steroid responder and poor steroid responder groups.

As a result, the RING6 gene and HLA-DMB gene were selected as a gene showing a decrease of ⅓ or less in the expression level in the steroid responder group. The expression level of the RING6 gene and HLA-DMB gene increases in poor steroid responsive patients with allergic dermatitis, and the genes are closely associated with poor steroid responsive allergic diseases.

The expression levels of RING7 and HLA-DMA, which were thought to constitute a family together with these genes, did not change on the same DNA chip. RING6 and HLA-DMB were therefore thought to be genes specifically involved in the steroid responsiveness in the family.

### EXAMPLE 2

### Expression Level of RING6 Gene or HLA-DMB Gene in Peripheral Blood Mononuclear Cells and Atopic Dermatitis

For quantitative confirmation of the expression level of the RING6 gene or HLA-DMB gene selected in Example 1, quantitative PCR by ABI 7700 was further performed with PBMC as a specimen.

The changes in the expression of the RING6 gene or HLA-DMB gene, which had been considered associated with the pathophysiology of steroid responsive allergic diseases were analyzed in mononuclear cells isolated from peripheral blood (peripheral blood mononuclear cells, PBMC) of atopic dermatitis patients and normal healthy subjects. 7 normal healthy volunteers, 5 responders to steroid ointment therapy and 6 poor-responders thereto were used as subjects. Isolation and culture of PBMC (peripheral blood mononuclear cell) and extraction of RNA for quantification of the gene expression level in this Example were carried out according to the methods as described in Example 1 (1). Operation of reverse transcription reaction and quantitative PCR method were performed as described below.

(1) DNase Treatment of Total RNA

The total RNA solution (20 μg), 10×DNase Buffer (5 μl) (Nippon Gene), RNase inhibitor (Amersham, Pharmacia Biotech) (25 units) and DNase I (Nippon Gene) (1 unit) were mixed and DNase and RNase-free water was added to a final volume of 50 μl. After incubation at 37° C. for 15 min, water-saturated phenol (pH 8.0) and CHCl₃ (25 μl each) were added to the mixture and mixed by tumbling. After centrifuging at 15,000 rpm at room temperature for 15 min, 3 M sodium acetate (pH 5.2) (5 μl), ethanol (125 μl) and Ethachinmate (1 μl) were added to the supernatant, and the resulting mixture was left standing at −20° C. for 15 min. After centrifuging at 15,000 rpm at 4° C. for 15 min, 80% ethanol (125 μl) was added to the precipitate, and the mixture was centrifuged at 15,000 rpm at 4° C. for 5 min. The precipitate was air-dried or vacuum-dried for 2 to 3 min, and dissolved in RNase-free distilled water (10 μl) to measure its absorbance as an RNA solution.

(2) Reverse Transcription Reaction

The RNA solution (1 to 5 μg), Oligo (dT)₁₂₋₁₈ primer (GIBCO BRL) (500 ng) and BSA (1 μg) were mixed and adjusted to a final volume of 12 μl with sterilized distilled water. The mixture was left standing at 70° C. for 10 min, and then cooled on ice. 5×First Strand Buffer (GIBCO BRL) (4 μl), 1 M DTT (2 μl) and 10 mM dNTPs (1 μl) (N=G, A, T, C) were added to the mixture and mixed. After heating the mixture at 42° C. for 2 min, SuperScriptII (GIBCO BRL) (200 units) was added thereto, and the mixture was reacted at 42° C. for 50 min. Then, the mixture was treated at 70° C. for 15 min to inactivate the reverse transcriptase. RNase H (GIBCO BRL) (2 units) was added thereto and incubated at 37° C. for 20 min. Sterilized distilled water was added to the mixture to prepare a cDNA solution of a concentration of 10 ng/μl and the solution was subjected to quantitative PCR.

(3) PCR Amplification of Target Region

10×PCR Buffer (100 mM Tris-HCl, pH 8.3, 500 mM KCl, 15 mM MgCl₂) (5 μl), 2.5 mM dNTPs (4 μl) (N=G, A, T, C), primer F (10 pmol/μl), primer R (10 pmol/μl), cDNA solution (5 ng) and rTaq DNA polymerase (TaKaRa) (1.25 units) were mixed and adjusted to a final volume of 50 μl with sterilized distilled water. The nucleotide sequences of the primers are as follows:

For RING6 gene amplification,

primer F: 5′-TGC GCT GCT ACA GAT GTT ACC-3′/SEQ ID NO: 2; and

primer R: 5′-CTG TGT GCA GGA ATG TGT GGT-3′/SEQ ID NO: 3.

For HLA-DMB gene amplification,

primer F: 5′-CAG AAG TGA CTA TCA CGT GGA GG-3′/SEQ ID NO: 4; and

primer R: 5′-AAA TGG GAG AGG GTC TGG TAT G-3′/SEQ ID NO: 5.

After the mixture was left standing at 95° C. for 10 min, 40 cycles of “95° C. for 15 s and 60° C. for 1 min” were carried out. Then, electrophoresis on 3% agarose gel (Agarose-1000, GIBCO-BRL)/5 μg/ml ethidium bromide in electrophoresis buffer solution 1×TAE (50×TAE contains Tris base (242 g), glacial acetic acid (57.1 ml) and 50 mM EDTA (pH 8.0) in 1 liter) at 100 V for 30 min was conducted. Then, the gel was scanned under an UV lamp to observe the band for a PCR product of 116 bp (RING6) or 112 bp (HLA-DMB).

(4) Excision of DNA Fragments

The PCR product of interest was excised from the gel using QIAEX II Agarose Gel Extraction kit (QIAGEN) according to the accompanying manual. After the isolation of the PCR products by electrophoresis on a 3% agarose gel, the fragment of interest was excised under a long wavelength (316 nm) UV. The gel was macerated using a razor, and transferred into a 1.5-ml tube (˜250 mg gel). 6 volumes of Buffer QX1 (300 μl for excised gel 50 mg) and QIAEX II glass bead (10 μl) were added and the mixture was thoroughly mixed for 30 s using a vortex mixer. The resulting mixture was heated at 50° C. for 10 min with mixing at several minutes' intervals until the mixture became yellow. When the color of the mixture was orange or purple, 3 M sodium acetate (pH 5.0) (10 μl) was added. After centrifugation at 12,000 rpm at room temperature for 30 s, Buffer QX1 (500 μl) was added to the precipitate, thoroughly vortexed, and the mixture was centrifuged at room temperature and 12,000 rpm for 30 s. Then, PE solution (500 μl) was added to the precipitate, and centrifuged at room temperature at 12,000 rpm for 30 sec (process (A)). The process (A) was repeated twice. Then, the supernatant was discarded and the precipitate was dried until it became white. Sterilized distilled water (20 μl) was added to the precipitate, and after leaving standing for 5 min, the mixture was centrifuged at room temperature at 12,000 rpm for 30 sec to recover the supernatant (process (B)). After repeating process (B) twice, the supernatant was subjected to agarose gel electrophoresis to confirm the extraction of the PCR product.

(5) TA Cloning of PCR Product

Cloning of the purified PCR product was conducted using a pGEM^{R}-T Easy Vector System I (Promega) according to the accompanying manual. 2×Rapid Ligation Buffer (5 μl), pGEM^{R}-T Easy Vector (50 ng/μl) (1 μl), the purified PCR product (3 μl) and T4 DNA Ligase (3 Weiss units/μl) (1 μl) were mixed and left standing at room temperature for 1 h (or at 16° C. overnight). Ligation reaction solution (2 μl) was added to Competent Cells DH5α (GIBCO BRL) (50 μl), and the resulting mixture was left on ice for 20 min. Then, heat shock treatment at 42° C. for 45 to 50 sec was conducted, and the treated mixture was left standing on ice for 2 min. SOC medium (GIBCO BRL) (950 μl) was added to the cells and mixed at 37° C. for 1 to 1.5 h at 150 rpm. The cell culture (100 μl) was plated on LB/amp/IPTG/X-gal and left standing at 37° C. overnight.

(6) Plasmid DNA Extraction

The subcloned plasmid DNA was extracted using Wizard Plus SV Minipreps DNA Purification System (Promega) according to the accompanying manual. First, white colonies were picked up, cultured in ampicillin (100 μg/ml)-LB medium (1 to 5 ml) at 37° C. overnight, and then centrifuged at 3,000 rpm for 6 min. Resuspended solution (250 μl) was added to suspend the precipitate; Lysis solution (250 μl) was added thereto and mixed 4 times by tumbling. Alkaline protease (10 μl) was added thereto, mixed 4 times by tumbling and the mixture was left standing at room temperature for 5 min. Neutralization solution (350 μl) was added to the mixture, mixed 4 times by tumbling, and centrifuged at room temperature at 14,000 rpm for 10 min. Then, the supernatant was transferred on a column included in the kit by decantation and centrifuged at room temperature at 14,000 rpm for 10 min. 700 μl of wash solution was added to the column portion (the follow-through fraction was discarded), and the mixture was centrifuged at room temperature at 14,000 rpm for 1 min. Then, 250 μl of the wash solution was added to the column portion (the follow-through fraction was discarded), and the mixture was centrifuged at room temperature at 14,000 rpm for 2 min. The column portion was transferred into a fresh tube, sterilized distilled water (20 μl) was added thereto, and the mixture was centrifuged at room temperature at 14,000 rpm for 1 min. The obtained solution was used as a plasmid DNA preparation and its concentration was determined by absorbance measurement.

(7) Sequence Reaction

Sequence reaction for confirming whether the subcloned plasmid DNA contains the DNA sequence of interest or not was performed using Thermo Sequinase II dye terminator (Amersham Pharmacia Biotech) according to the accompanying manual. First, M13 primer (3 pmol), the DNA solution (200 to 300 ng) and TSII Reagent Mix (2 ρl) were mixed and adjusted to a final volume of 10 μl with sterilized distilled water. After leaving standing at 96° C. for 1 min, 30 cycles (96° C. for 30 sec, 50° C. for 15 sec, and 60° C. for 1 min as one cycle) were performed and then the temperature was lowered to 4° C. Then, 1.5 M sodium acetate/250 mM EDTA (1 μl) was added to the reaction solution and vortexed. Isopropanol (20 μl) was added, thoroughly mixed, and the mixture was left standing at room temperature for 10 min. After centrifugation at 12,000 rpm for 20 min, 70% ethanol (150 μl) was added to the precipitate and mixed. The mixture was then centrifuged at 12,000 rpm for 5 min, and the precipitate was air-dried or vacuum-dried for 2 to 3 min. Next, after the addition of loading dye (1.5 μl) to the dried precipitate, the mixture was subjected to a heat treatment at 95° C. for 2 min, and then cooled on ice. The whole reaction product was applied on a LongRanger gel [LongRanger (5 ml), urea (15 g), 10×TBE (5 ml), 10% APS (250 μl) and TEMED (35 μl), adjusted to a final volume of 50 ml with sterilized distilled water] set on ABI377 DNA sequencer (Applied Biosystems) to start electrophoresis. After confirming the PCR product to contain the objective DNA sequence, the product was used as the standard sample.

(8) Quantitative PCR

Quantification of the gene expression level was carried out by real-time PCR using ABI PRISM 7700 System with TaqMan probe according to the accompanying manual. TaqMan 1000 Reaction PCR Core reagents (Applied Biosystems) were used according to the accompanying manual as the reaction reagent. At least 5 gradients between 10⁷ to 10³ copies of the concentration gradient were prepared as the standard samples for plotting a calibration curve. The “n” number per one sample was set as at least 2. 10×Buffer A (5 μl), 25 mM MgCl₂ (7 μl), 10 mM dNTPs (1 μl each) (N=G, A, T, C), AmpTaqGold (1.25 units), UNG (0.5 units), primer F (10 pmol), primer R (10 pmol), cDNA solution (5 ng) and TaqMan Probe (5 pmol) were mixed and adjusted to a final volume of 50 μl with sterilized distilled water. As the primers for amplification, the same as in (3) (SEQ ID NOs: 2 and 3 for RNase A gene, and SEQ ID NOs: 4 and 5 for HLA-DMB gene amplification) were used and the probe had the following nucleotide sequence:

TaqMan probe for RING6 gene:
5′- (FAM) TCC TAC TCC AAT GTG GCC AGA
TGA CC -3′ (TAMRA)/SEQ ID NO: 6
TaqMan probe for HLA-DMB gene:
5′- (FAM) AAC GGG AAG CTT GTC ATG
CCT CAC A -3′ (TAMRA)/SEQ ID NO: 7
FAM: 6-carboxyfluorescein
TAMRA: 6-carboxy-tetramethylrhodamine

After leaving the above reaction mixture standing at 50° C. for 2 min and then at 95° C. for 10 min, 50 cycles (95° C. for 15 sec and 60° C. for 1 min as one cycle) were performed. A calibration curve was automatically plotted from the Ct (threshold cycles) value of PCR amplification curve plotted against the logarithm of relative initial concentrations of the standard sample. Then, based on the calibration curve, relative initial concentrations of cDNA in unknown samples were calculated.

In order to correct the difference in the cDNA concentrations among samples, a similar quantitative analyses were carried out for the β-actin gene and glyceraldehyde-3-phosphate dehydrogenase (GAPDH) gene as the internal standard for correction to calculate the copy number of the target gene based on their copy numbers.

As the primers and probes for the measurement of β-actin and GAPDH genes, those included in TaqMan β-actin Control Reagents (Applied Biosystems) were utilized. Their nucleotide sequences were as follows:

β-actin forward primer (SEQ ID NO: 8)
TCA CCC ACA CTG TGC CCA TCT ACG A;
β-actin reverse primer (SEQ ID NO: 9)
CAG CGG AAC CGC TCA TTG CCA ATG G;
β-actin TaqMan probe (SEQ ID NO: 10)
(FAM)ATGCCC-T(TAMRA)-CCCCCATGCCATCCTGCGTp-3′;
GAPDH forward primer (SEQ ID NO: 11)
GAAGGTGAAGGTCGGAGT;
GAPDH reverse primer (SEQ ID NO: 12)
GAAGATGGTGATGGGATTTC; and
GAPDH TaqMan probe (SEQ ID NO: 13)
(FAM)CAAGCTTCCCGTTCTCAGCC(TAMRA)-3′.

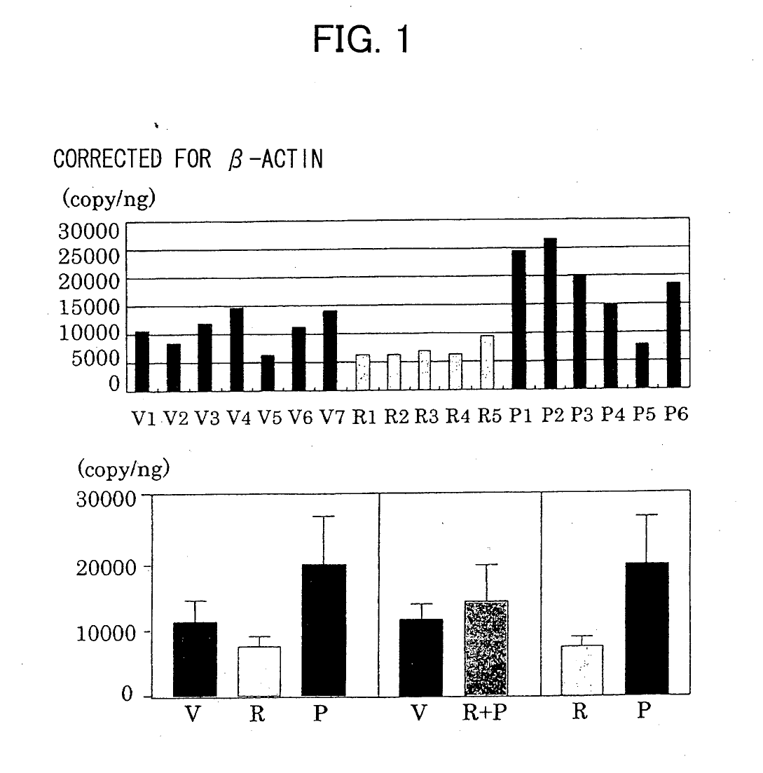

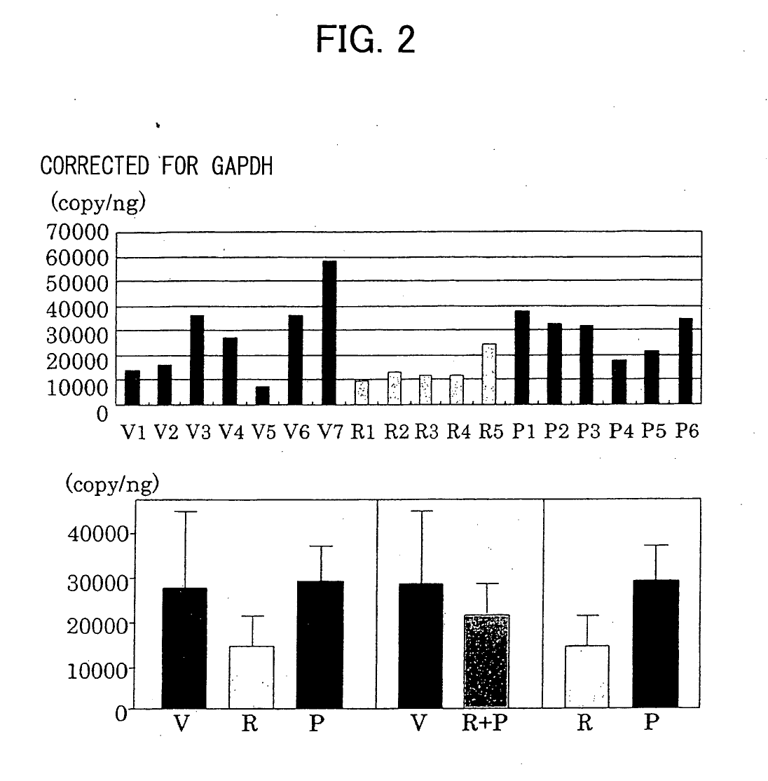

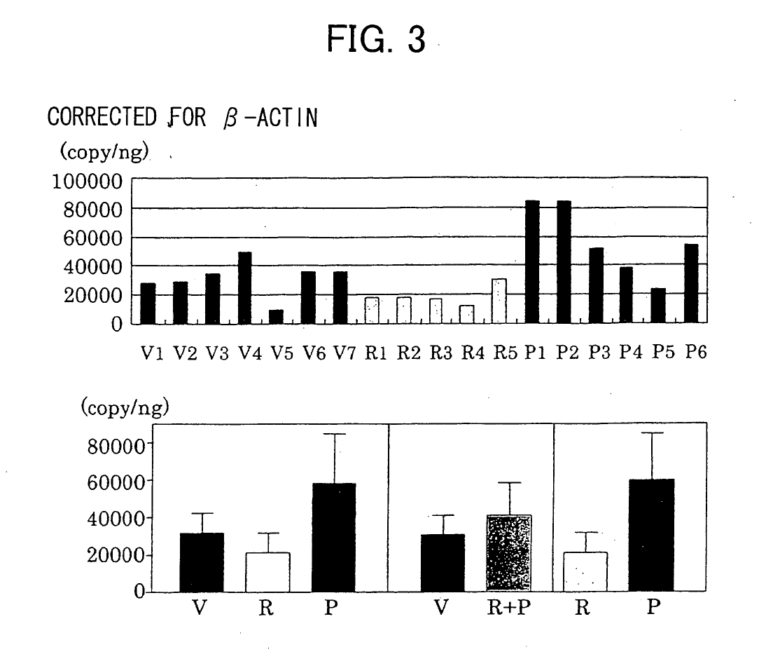

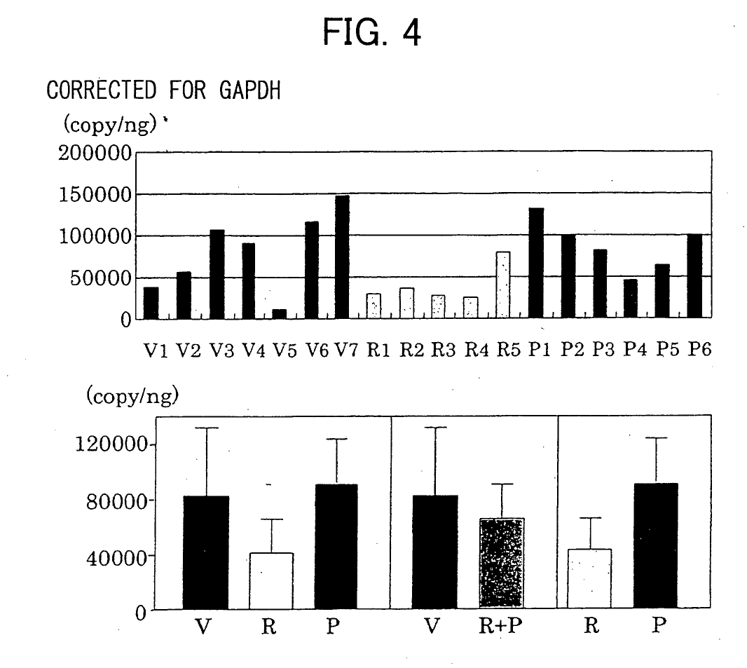

Measurement results are shown in Tables 2 (RING6) and 3 (HLA-DMB). Furthermore, based on the measured values, the expression level (copy/ng RNA) of the RING6 gene corrected for p-actin are shown in FIG. 1 (upper panel), and that corrected for GAPDH in FIG. 2 (upper panel). In addition, based on the measured values, the expression level (copy/ng RNA) of the HLA-DMB gene corrected for β-actin are shown in FIG. 3 (upper panel), and that corrected for GAPDH in FIG. 4 (upper panel).

TABLE 2

| | mRNA expression level (copy/ng) | | |
|---|---|---|---|
| | | Corrected for | Corrected for |
| Type | Raw data | β-actin | GAPDH |
| V1 | 5735 | 10725 | 14350 |
| V2 | 11612 | 8412 | 16267 |
| V3 | 9537 | 11768 | 36246 |
| V4 | 5718 | 14540 | 26944 |
| V5 | 8084 | 6324 | 7427 |
| V6 | 12124 | 11162 | 36102 |
| V7 | 13875 | 14191 | 58213 |
| R1 | 4126 | 6097 | 9880 |
| R2 | 3439 | 6294 | 12959 |
| R3 | 4705 | 6956 | 11740 |
| R4 | 2079 | 6267 | 12051 |
| R5 | 4475 | 9294 | 24434 |
| P1 | 8225 | 24238 | 37750 |
| P2 | 9044 | 26616 | 32117 |
| P3 | 4669 | 20048 | 31605 |
| P4 | 3464 | 14679 | 17497 |
| P5 | 8964 | 7960 | 21162 |
| P6 | 6975 | 18579 | 34323 |
| | copy number (mean ± SD) | | |
| | V (n = 7) | R (n = 5) | P (n = 6) |
| Rawdata | 9526 ± 3188 | 3765 ± 1057 | 6890 ± 2341 |
| Corrected for | 11017 ± 2943 | 6982 ± 1334 | 18687 ± 6733 |
| β-actin | | | |
| Corrected for | 27936 ± 17295 | 14213 ± 5823 | 29076 ± 7939 |
| GAPDH | | | |

TABLE 3

| | mRNA expression level (copy/ng) | | |
|---|---|---|---|
| | | Corrected for | Corrected for |
| Type | Raw data | β-actin | GAPDH |
| V1 | 15075 | 28192 | 37718 |
| V2 | 40936 | 29654 | 57344 |
| V3 | 28221 | 34822 | 107253 |
| V4 | 19355 | 49214 | 91199 |
| V5 | 13379 | 10467 | 12292 |
| V6 | 38663 | 35594 | 115124 |
| V7 | 35444 | 36251 | 148707 |
| R1 | 12020 | 17761 | 28783 |
| R2 | 9626 | 17613 | 36268 |
| R3 | 11107 | 16419 | 27711 |
| R4 | 4276 | 12892 | 24791 |
| R5 | 14679 | 30484 | 80142 |
| P1 | 28526 | 84062 | 130923 |
| P2 | 28479 | 83814 | 101136 |
| P3 | 12157 | 52199 | 82287 |
| P4 | 8965 | 37994 | 45287 |
| P5 | 26596 | 23617 | 62790 |
| P6 | 20309 | 54093 | 99935 |
| | copy number (mean ± SD) | | |
| | V (n = 7) | R (n = 5) | P (n = 6) |
| Raw data | 27296 ± 11464 | 10342 ± 3857 | 20839 ± 8570 |
| Corrected for | 32028 ± 11683 | 19034 ± 6695 | 55963 ± 24298 |
| β-actin | | | |
| Corrected for | 81377 ± 47775 | 39539 ± 23089 | 87060 ± 30478 |
| GAPDH | | | |

(9) Statistical Analysis

The statistical analysis of 7 healthy normal volunteers (V group), 5 responders to steroid ointment treatment (R group) and 6 poor-responders to said treatment (P group) were performed by the Fisher's analysis of variance (ANOVA) and the Kruskal-Walli test for the comparisons among 3 groups, and the comparisons between 2 groups, either between normal (V) and patient (R+P) groups, or between responder (R) and poor-responder (P) groups were performed by the Fisher's analysis of variance and the Mann-Whitney test. Analytical results of responders (R) and poor responders (P) are shown in Tables 4 (RING6) and 5 (HLA-DMB), respectively. Furthermore, comparison results of the two groups with healthy normal subjects (V) are shown in FIG. 1 (lower panel) and FIG. 2 (lower panel)/RING6 gene, and FIG. 3 (lower panel) and FIG. 4 (lower panel)/HLA-DMB gene, respectively.

TABLE 4

| Comparison between P/R two | | ANOVA | Mann-Whitney |
|---|---|---|---|
| groups | Difference | p value | p value |
| Raw data | P > R | 0.0027 | 0.0446 |
| Corrected for β-actin | P > R | 0.0043 | 0.0106 |
| Corrected for GAPDH | P > R | 0.0071 | 0.0176 |

TABLE 5

| Comparison between P/R two | | ANOVA | Mann-Whitney |
|---|---|---|---|
| groups | Difference | p value | p value |
| Raw data | P > R | 0.0329 | 0.0679 |
| Corrected for β-actin | P > R | 0.0097 | 0.0106 |
| Corrected for GAPDH | P > R | 0.0188 | 0.0176 |

As judged from the data obtained by the quantitative PCR, the expression level of the RING6 gene or the HLA-DMB gene selected in Example 1 in mononuclear cells was reduced to ½ or below in the steroid responder group as compared to the poor responder group. Furthermore, no significant difference was observed between the poor steroid responder group and healthy normal subjects. Based on these results, one may conclude that the elevation of expression level of the RING6 gene or HLA-DMB gene in mononuclear cells can serve as a marker for poor steroid responsiveness in patients with allergic diseases.

Industrial Applicability

The present invention reveals genes with a decreased expression level in mononuclear cells in a steroid responder group. These genes may serve as markers for responsiveness to steroids in allergic dermatitis patients. Furthermore, the marker genes of the present invention are expected to be useful as markers for Th1 cell decrease.

The decrease in the expression level of the marker genes of the present invention is associated with responsiveness to steroids. Thus, suppression of the expression level of the genes serves as a target of therapeutic strategy for disorders for which steroid administration is selected as a treatment. Furthermore, the genes are also expected to be useful as novel clinical diagnostic markers for monitoring the effect of such new therapeutic methods. Allergic diseases are typical examples of such disorders. Alternatively, administration of an anti-sense drug against the genes or antibodies inhibiting the activity of the proteins to suppress the elevation in the expression level or activity of the translation products may function as a therapeutic method for allergic diseases.

Since the method for testing steroid responsiveness of this invention enables the analysis of the expression level of a marker gene using a biological specimen as a test sample, it is less invasive to patients. Furthermore, gene expression analyses facilitate highly sensitive measurement of gene expression in minute quantities of test samples. Year by year, gene analytical techniques are being improved for higher throughput and prices are being reduced. Therefore, the method for testing steroid responsiveness according to the present invention is expected to become an important bedside diagnostic method in the near future. In this regard, the gene associated with steroid responsiveness is highly valuable in diagnosis.

1. A method for testing steroid responsiveness, comprising the steps of:
a) measuring the expression level of the RING6 gene or HLA-DMB gene in a biological sample of a test subject; and
b) comparing the measured expression level to that of the same gene in a biological sample taken from either a normal healthy subject or poor steroid responsive subject.

2. The method according to claim 1, wherein the steroid responsiveness of an allergic disease is tested.

3. The method according to claim 2, wherein the allergic disease is atopic dermatitis.

4. The method according to claim 1, wherein the expression level of the gene is measured by PCR of cDNA.

5. The method according to claim 1, wherein the expression level of the gene is measured by detecting the protein encoded by the gene.

6. A reagent for testing steroid responsiveness, said reagent comprising an oligonucleotide having a nucleotide sequence complementary to a polynucleotide comprising the nucleotide sequence of the RING6 gene or HLA-DMB gene or to the complementary strand thereof, which oligonucleotide has a length of at least 15 nucleotides.

7. A reagent for testing steroid responsiveness, said reagent comprising an antibody recognizing a peptide containing the amino acid sequence of the RING6 protein or HLA-DMB protein.

8. A method of screening for a compound that elevates steroid responsiveness, comprising the steps of:
(1) contacting a candidate compound with a cell that expresses a gene selected from the group consisting of the RING6 gene, the HLA-DMB gene and genes functionally equivalent thereto;
(2) measuring the expression level of the gene; and
(3) selecting the compound that reduces the expression level of the gene as compared to the expression level associated with a control cell that has not been contacted with the candidate compound.

9. The method according to claim 8, wherein the cell is a mononuclear cell line.

10. A method of screening for a compound that elevates steroid responsiveness, comprising the steps of:
(1) administering a candidate compound to a test animal;
(2) measuring the expression intensity in a biological sample from the test animal of a gene selected from the group consisting of the RING6 gene, the HLA-DMB gene and genes functionally equivalent thereto; and
(3) selecting the compound that reduces the expression level of the gene as compared to the expression level associated with a control animal not administered the candidate compound.

11. A method of screening for a compound that elevates steroid responsiveness, comprising the steps of:
(1) contacting a candidate compound with a cell transfected with a vector comprising a transcriptional regulatory region of a gene selected from the group consisting of the RING6 gene; the HLA-DMB gene and genes functionally equivalent thereto, and a reporter gene that is expressed under the control of the transcriptional regulatory region;
(2) measuring the activity of the reporter gene; and
(3) selecting the compound that reduces the expression level of the gene as compared to the expression level associated with a control cell that has not been contacted with the candidate compound.

12. A method of screening for a compound that elevates steroid responsiveness, comprising the steps of:
(1) contacting a candidate compound with a protein selected from the group consisting of the RING6 protein, the HLA-DMB protein and proteins functionally equivalent thereto;
(2) measuring the activity of the protein; and
(3) selecting the compound that reduces the activity of the protein compared to the activity associated with a control protein that has not been contacted with the candidate compound.

13-19. (canceled)

20. A pharmaceutical composition that elevates steroid responsiveness, which comprises as an effective ingredient a compound obtained by the method according to claim 8.

21. A pharmaceutical composition that elevates steroid responsiveness, which comprises as an effective ingredient a compound obtained by the method according to claim 10.

22. A pharmaceutical composition that elevates steroid responsiveness, which comprises as an effective ingredient a compound obtained by the method according to claim 11.

23. A pharmaceutical composition that elevates steroid responsiveness, which comprises as an effective ingredient a compound obtained by the method according to claim 12.

24. A pharmaceutical composition that elevates steroid responsiveness, which comprises as the primary active ingredient an anti-sense DNA against the RING6 gene, the HLA-DMB gene or a portion thereof.

25. A pharmaceutical composition to elevate steroid responsiveness, which comprises as the primary active ingredient an antibody recognizing a peptide comprising an amino acid sequence of the RING6 protein or the HLA-DMB protein.

26. A therapeutic agent for poor steroid responsive disorders comprising the pharmaceutical according to claim 20 in combination with a steroid drug.

27. A therapeutic agent for poor steroid responsive disorders comprising the pharmaceutical according to claim 21 in combination with a steroid drug.

28. A therapeutic agent for poor steroid responsive disorders comprising the pharmaceutical according to claim 22 in combination with a steroid drug.

29. A therapeutic agent for poor steroid responsive disorders comprising the pharmaceutical according to claim 23 in combination with a steroid drug.

30. A therapeutic agent for poor steroid responsive disorders comprising the pharmaceutical according to claim 24 in combination with a steroid drug.

31. A therapeutic agent for poor steroid responsive disorders comprising the pharmaceutical according to claim 25 in combination with a steroid drug.

32. A kit for screening a candidate compound for a therapeutic agent for an allergic disease, said kit comprising an oligonucleotide containing at least 15 nucleotides, wherein the oligonucleotide is complementary to a polynucleotide comprising the nucleotide sequence of the RING6 gene, the HLA-DMB gene or the complementary strand thereof, and a cell expressing the RING6 gene or HLA-DMB gene.

33. A kit for screening a candidate compound for a therapeutic agent for an allergic disease, said kit comprising an antibody recognizing a peptide containing the amino acid sequence of the RING6 protein or HLA-DMB protein, and a cell expressing the RING6 gene or HLA-DMB gene.

34. Use of a transgenic non-human vertebrate in which the expression intensity of a gene selected from the group consisting of the RING6 gene, the HLA-DMB gene, and genes functionally equivalent thereto in mononuclear cells is regulated as a steroid responsiveness-regulated model.


SEQUENCE LISTING (19 sequences)

SEQ ID NO: 1 - DNA, 63 bp, Artificial Sequence
  Description of Artificial Sequence: an artificially synthesized T7-(dT)24 primer sequence
ggccagtgaattgtaatacgactcactatagggaggcggtttttttttttttttttttttttt

SEQ ID NO: 2 - DNA, 21 bp, Artificial Sequence
  Description of Artificial Sequence: an artificially synthesized primer sequence
tgcgctgctacagatgttacc

SEQ ID NO: 3 - DNA, 21 bp, Artificial Sequence
  Description of Artificial Sequence: an artificially synthesized primer sequence
ctgtgtgcaggaatgtgtggt

SEQ ID NO: 4 - DNA, 23 bp, Artificial Sequence
  Description of Artificial Sequence: an artificially synthesized primer sequence
cagaagtgactatcacgtggagg

SEQ ID NO: 5 - DNA, 22 bp, Artificial Sequence
  Description of Artificial Sequence: an artificially synthesized primer sequence
aaatgggagagggtctggtatg

SEQ ID NO: 6 - DNA, 26 bp, Artificial Sequence
  Description of Artificial Sequence: an artificially synthesized TaqMan probe sequence
  misc_binding (1)OTHER INFORMATION: Label FAM (6-carboxyfluorescei    n)
  misc_binding (26): Label TAMRA (6-carboxy-tetramethylrhodamine)
tcctactccaatgtggccagatgacc

SEQ ID NO: 7 - DNA, 25 bp, Artificial Sequence
  Description of Artificial Sequence: an artificially synthesized TaqMan probe sequence
  misc_binding (1): Label FAM (6-carboxyfluorescein)
  misc_binding (25): Label TAMRA (6-carboxy-tetramethylrhodamine)
aacgggaagcttgtcatgcctcaca

SEQ ID NO: 8 - DNA, 25 bp, Artificial Sequence
  Description of Artificial Sequence: an artificially synthesized primer sequence
tcacccacactgtgcccatctacga

SEQ ID NO: 9 - DNA, 25 bp, Artificial Sequence
  Description of Artificial Sequence: an artificially synthesized primer sequence
cagcggaaccgctcattgccaatgg

SEQ ID NO: 10 - DNA, 26 bp, Artificial Sequence
  Description of Artificial Sequence: an artificially synthesized TaqMan probe sequence
  misc_binding (1): Label FAM (6-carboxyfluorescein)
  misc_binding (7): Label TAMRA (6-carboxy-tetramethylrhodamine)
atgccctcccccatgccatcctgcgt

SEQ ID NO: 11 - DNA, 18 bp, Artificial Sequence
  Description of Artificial Sequence: an artificially synthesized primer sequence
gaaggtgaaggtcggagt

SEQ ID NO: 12 - DNA, 20 bp, Artificial Sequence
  Description of Artificial Sequence: an artificially synthesized primer sequence
gaagatggtgatgggatttc

SEQ ID NO: 13 - DNA, 20 bp, Artificial Sequence
  Description of Artificial Sequence: an artificially synthesized TaqMan probe sequence
  misc_binding (1): Label FAM (6-carboxyfluorescein)
  misc_binding (20): Label TAMRA (6-carboxy-fluorescein)
caagcttcccgttctcagcc

SEQ ID NO: 14 - DNA, 1100 bp, Homo sapiens
  (46)..(831)
ctaaagctgggttggtagctcctacctactgtgtggcaagaaggtatgggtcatgaacagaaccaaggagctgcgctgctacagatgttaccacttctgtggctgctaccccactcctgggccgtccctgaagctcctactccaatgtggccagatgacctgcaaaaccacacattcctgcacacagtgtactgccaggatgggagtcccagtgtgggactctctgaggcctacgacgaggaccagcttttcttcttcgacttttcccagaacactcgggtgcctcgcctgcccgaatttgctgactgggctcaggaacagggagatgctcctgccattttatttgacaaagagttctgcgagtggatgatccagcaaatagggccaaaacttgatgggaaaatcccggtgtccagagggtttcctatcgctgaagtgttcacgctgaagcccctggagtttggcaagcccaacactttggtctgttttgtcagtaatctcttcccacccatgctgacagtgaactggcacgatcattccgtccctgtggaaggatttgggcctacttttgtctcagctgtcgatggactcagcttccaggccttttcttacttaaacttcacaccagaaccttctgacattttctcctgcattgtgactcacgaaattgaccgctacacagcaattgcctattgggtaccccggaacgcactgccctcagatctgctggagaatgtgctgtgtggcgtggcctttggcctgggtgtgctgggcatcatcgtgggcattgttctcatcatctacttccggaagccttgctcaggtgactgattcttccagaccagagtttgatgccagcagcttcggccatccaaacagaggatgctcagatttctcacatcctgcccaggatctcctcttagggtagaagaagtctctgggacatccctggggtgtgtgtgtagatttcccacctggggactctgctgtccctgggcttgcatcccagggatcccagagtggcctgcctatcacaaccacatcccttccccccacaaggcaataaatctcatttctttaaaaaaaaaaaaaaaaaaaaa

SEQ ID NO: 15 - protein, 261 aa, Homo sapiens
MGHEQNQGAALLQMLPLLWLLPHSWAVPEAPTPMWPDDLQNHTFLHTVYCQDGSPSVGLSEAYDEDQLFFFDFSQNTRVPRLPEFADWAQEQGDAPAILFDKEFCEWMIQQIGPKLDGKIPVSRGFPIAEVFTLKPLEFGKPNTLVCFVSNLFPPMLTVNWHDHSVPVEGFGPTFVSAVDGLSFQAFSYLNFTPEPSDIFSCIVTHEIDRYTAIAYWVPRNALPSDLLENVLCGVAFGLGVLGIIVGIVLIIYFRKPCSGD

SEQ ID NO: 16 - DNA, 1362 bp, Homo sapiens
  CDS (234)..(1025)
cctgtttgggacactggactcccgtgagctggaaggaacagatttaatatctaggggctgggtatccccacatcactcatttggggggtcaagggacccgggcaatatagtattctgctcagtgtctggagatcatctacccaggctggggcttctgggacaggcgaggacccacggaccctggaagagctggtccaggggactgaactcccggcatctttacagagcagagcatgatcacattcctgccgctgctgctggggctcagcctgggctgcacaggagcaggtggcttcgtggcccatgtggaaagcacctgtctgttggatgatgctgggactccaaaggatttcacatactgcatctccttcaacaaggatctgctgacctgctgggatccagaggagaataagatggccccttgcgaatttggggtgctgaatagcttggcgaatgtcctctcacagcacctcaaccaaaaagacaccctgatgcagcgcttgcgcaatgggcttcagaattgtgccacacacacccagcccttctggggatcactgaccaacaggacacggccaccatctgtgcaagtagccaaaaccactccttttaacacgagggagcctgtgatgctggcctgctatgtgtggggcttctatccagcagaagtgactatcacgtggaggaagaacgggaagcttgtcatgcctcacagcagtgcgcacaagactgcccagcccaatggagactggacataccagaccctctcccatttagccttaaccccctcttacggggacacttacacctgtgtggtagagcacattggggctcctgagcccatccttcgggactggacacctgggctgtcccccatgcagaccctgaaggtttctgtgtctgcagtgactctgggcctgggcctcatcatcttctctcttggtgtgatcagctggcggagagctggccactctagttacactcctcttcctgggtccaattattcagaaggatggcacatttcctagaggcagaatcctacaacttccactccaagtgagaaggagattcaaactcaatgatgctaccatgcctctccaacatcttcaaccccctgacattatcttggatcctatggtttctccatccaattctttgaatttcccagtctcccctatgtaaaacttagcaacttgggggacctcattcctgggactatgctgtaaccaaattattgtccaaggctatatttctgggatgaatataatctgaggaagggagttaaagaccctcctggggctctcagtgtgccatagaggacagcaactggtgattgtttcagagaaataaactttggtggaaaaa

SEQ ID NO: 17 - protein, 263 aa, Homo sapiens
MITFLPLLLGLSLGCTGAGGFVAHVESTCLLDDAGTPKDFTYCISFNKDLLTCWDPEENKMAPCEFGVLNSLANVLSQHLNQKDTLMQRLRNGLQNCATHTQPFWGSLTNRTRPPSVQVAKTTPFNTREPVMLACYVWGFYPAEVTITWRKNGKLVMPHSSAHKTAQPNGDWTYQTLSHLALTPSYGDTYTCVVEHIGAPEPILRDWTPGLSPMQTLKVSVSAVTLGLGLIIFSLGVISWRRAGHSSYTPLPGSNYSEGWHIS

SEQ ID NO: 18 - DNA, 2100 bp, Homo sapiens
gatcaggctctttctctctgtaaacctgctcccatggcattcttttctgcttcaattcagtccatccctgaccacccacccagccacccatgctggaaatctgagatatctttgactcctccaccttcccactcccacagctaatcagtagccaaaccctttagattctacttcctaattatcttgatattctatctcctctgcctcctctaattatgacttaattcaggctcttattatctgtcatttaaattattgctgtattgctacaggagtggtgtgtatatgtgcaactgttcatcttggtatgctatagactcttgcccagcagttttgcctcaattctttaaaagctccagttcattgtttgaaatatcaatcagaacattttttccaaaatgctaaaatgattgtcattccccagttttaatatctctatcatctgctaagtaaaagcaagctcctccttgtggcgtagcagggcccacgtgatctggcccttgcccgaatttgtagcttatcacaacatccttctaccccccattacctcctattatacaggaacacccaagcgaccttcacaacattcatatcaactagtacacagggtctccaaagaggacctctggatgcctgtgatctttgtacttctgcctaccctttgccctggcgtgtttttctcttccttcatgttctgctggccaaattgtttttccttcaagtcttaggagcctgcctgattatgttcctcctattccgcatcctcataaaattactgcgacttccaaaacacttggcacattcttatttgaatacattacattgttgtgtaattatcccttggccccctcccccagcactagactctaagctctttgaaggcagactatgcctattcatctttgtatttctagccctgggcccaacacccagaatttcttgtattttcaatatatgtctgccaaatgaattagagctaatgcctgaaggagaataagtttttttgagaaagaggaagagccaggagggaggagaaggcagggtccgaagagccctgggggtttacttggggaagatgcggttccccattgcagcattctgccaggaggtggcactgctctgaagagccagctggtcgcttacagactgaggctgacagcttgaaaccaaagagggaactccaccaggaagcaacattcctccactgggcttcccagcccagttacatgccatactctgccctggtcaaacagccaagtcttcaggaggttactggccccaggcgtctccccagtgactgatgatgttaaaccctacgcttctctgattggtttagacaaaatgacaagggcacctattggaaatgatctggcaaaacatgatctaaggccaccctctcggggagggagttggggaagctgggttggctgggttggtagctcctacctactgtgtggcaagaaggtatgggtcatgaacagaaccaaggagctgcgctgctacagatgttaccacttctgtggctgctaccccactcctgggccgtccctgaaggtaagatggtactcctatttacttccatcctgaacctagggagcccactcagctttgtgaggaaaagcgctgtgctttgtgagtggtgggaagtcttatgaggcagtggaaacaacaggggagtggggaaagacagctgctatgtgtggttggtaaacgatatcagtgatgctttgcatgttccatttaagacaatctcttgaagcggagattgttctctccattttacaaatgaagaaaatgagactcagagttatttgtgcaagttcacaccattggaaagtggtagagctgggatttgaacaaagtgaatgttattttcactcctccactcaagattctactctgctttctatcattaacttatcttgtgattcttgaaaagtgtcttagtttccttctctgtagaggatggggcagaatggaagaataatctggaagatccctcttcctccaaatgtctgtttttctgggtattgggttaaaggtttcctgctggctgagcgattcctgccaagagcttcaaagaagagggattattttatg

SEQ ID NO: 19 - DNA, 1020 bp, Homo sapiens
acaatggaggcccatggagggattttccaggcccctattcctgatctccatggtggcgctgcttcccttattctgcagtcttgctttgagaccagacaattctgtactctgtctgtgccttcttccttctcagcagccctgcctctgccacctgccccagaagggctctgggtcctctcatctcaggacagacaagggtttttgtaattatcttcttgggtaacctccctgtaaggaataggagaggttatctggtcctggtcttttgggagagcattgagaagaaaaagttaaaccagtgggcagcccaggctgcatacatcctgggaatattgcgattattctctccagtaatctacggaaatctactggttgttctgcagaaaaagaattaagagagacaaggagacctgttgtctaagactaaggcaggatgacgtttacctagtaactgatgatgctaggctgaggcactcagtgatttgtctctacatttgtccctgcctacctagccaatctgtccctgtttgggacactggactcccgtgagctggaaggaacagatttaatatctaggggctgggtatccccacatcactcatttggggggtcaagggacccgggcaatatagtattctgctcagtgtctggagatcatctacccaggctggggcttctgggacaggcgaggacccacggaccctggaagagctggtccaggggactgaactcccggcatctttacagagcagagcatgatcacattcctgccgctgctgctggggctcagcctgggctgcacaggagcaggtaaggacacttcttctggggactctcccttcccctgctcctgtttcagggtaagggtgttccgttttgtaattgcatttgacaccccagatagtttgtctccctggtatacattcctatagcactttgtactttgtagcaattttaatgtaattaatctgtataattatctgtgcagtgtatattccctgctggaatataggcaaggac